(19) United States
(12) Patent Application Publication
(10) Pub. No.: US 2024/0197385 A1
(43) Pub. Date: Jun. 20, 2024

(54) METHODS AND APPARATUS FOR PERFORMING ABLATION OF TISSUE

(71) Applicant: AtriCure, Inc., Mason, OH (US)

(72) Inventors: Robert James Klein, Lebanon, OH (US); Wanran Ma, Mason, OH (US); Christopher Lewis Gillum, Maineville, OH (US); Salvatore Privitera, Mason, OH (US)

(73) Assignee: AtriCure, Inc., Mason, OH (US)

(21) Appl. No.: 18/227,632

(22) Filed: Jul. 28, 2023

Related U.S. Application Data
(60) Provisional application No. 63/373,370, filed on Aug. 24, 2022.

(51) Int. Cl. A61B 18/14; A61B 18/00; A61B 18/18

(52) U.S. Cl. CPC A61B 18/14; A61B 2018/00083; A61B 2018/0016; A61B 2018/00178; A61B 2018/00577; A61B 2018/00702; A61B 2018/1467; A61B 2018/1823

(57) ABSTRACT

An electrosurgical system comprising an electrosurgical device, including a first electrode, a second electrode, and an intermediate electrical element, where the first electrode, the second electrode, and the intermediate electrical element are configured to electrically communicate with a target tissue, and where the intermediate electrical element interposes the first electrode and the second electrode, the system also including a resistive voltage divider electrically connected to a first input conductor and a second input conductor, the resistive voltage divider comprising a first resistor and a second resistor, where the first resistor and the second resistor are electrically connected in series between the first input conductor and the second input conductor, and where the first electrode is configured to electrically connect to the first input connector, the second electrode is configured to electrically connect to the second input connector, and the at least one intermediate electrode is configured to electrically connect to at least one intermediate conductor electrically connected between the first resistor and the second resistor.

### CROSS REFERENCE TO RELATED APPLICATIONS

This application claims the benefit of U.S. Provisional Application No. 63/373,370, filed Aug. 24, 2022, titled “METHODS AND APPARATUS FOR PERFORMING DEEP ABLATIONS FOR RADIOFREQUENCY ABLATION OF TISSUE,” which is incorporated herein by reference in its entirety.

### INTRODUCTION

The present disclosure is directed to medical devices and related methods, and, more specifically, to electrosurgical devices and methods, such as devices for ablating tissue and related methods.

The present disclosure contemplates that ablation (e.g., radiofrequency (RF) ablation) can be used as part of surgical procedures to treat atrial fibrillation, an irregular and rapid heart rate. RF ablation heats heart tissue to create lesions which interfere with abnormal electrical signals, which may be part of the process of restoring a normal heartbeat. Specific patterns of ablation can redirect electrical signals into more appropriate patterns in order to help treat atrial fibrillation. RF ablation can be applied by clamping tissue via an RF clamp or by pressing and/or suctioning on the exterior of tissue via an RF pen and/or suction device. RF ablation is typically performed at 1V-100V, 100 kHz-1000 kHz, and 1 W-100 W total power output. Tissue thickness in critical regions of the heart can range from 2 mm-15 mm. Tissue is preferably ablated to achieve transmurality, such that the ablation reaches essentially all the way through the thickness of the heart tissue muscle.

The present disclosure contemplates that ablation of tissue can be achieved by heating the tissue to about 55° C. to 60° C. However, various factors during ablation can often result in tissue being heated beyond this range (e.g., to 100° C. or higher). Overheating of the tissue can lead to drawbacks such as surface damage and/or charred tissue (which can affect healing time) and steam pops (which can cause holes in tissue). Additionally, some end effectors can reach high temperatures after one or more ablations as heat is generated at the tissue. This can result in parts of the end effector being heated above 55° C., which can result in damage to regions in the body that are adjacent to the heart and contact the end effector.

The present disclosure contemplates that some strategies to cool the heart tissue include adding heat sinks in the device end effector, including active cooling liquid, and reducing electrical output of the device. However, these methods may have their own drawbacks. For example, heat sinks can become excessively hot after multiple ablations. Active cooling requires a source of liquid from the exterior of the device, adding complications to devices and requiring more energy for ablation. Reducing electrical output can result in decreased efficiency of the procedure.

Therefore, although known devices have been used safely and effectively, there exists a need to provide improved devices and methods that can improve electrosurgery (e.g., ablation) of tissue while limiting unnecessary damage to the ablated tissue or surrounding tissue.

The following introduces some aspects of the present disclosure to achieve a basic understanding of the discussed technology. This introduction is not an extensive overview of all contemplated features of the disclosure and is intended neither to identify key or critical elements of all aspects of the disclosure nor to delineate the scope of any or all aspects of the disclosure. Its sole purpose is to present one or more aspects of the disclosure in introductory form as a prelude to the more detailed description that is presented later.

It is an aspect of the present disclosure to provide an ablation device for use with an energy source to apply energy to a tissue, the ablation device comprising: (a) an end effector having a working surface; (b) a connector configured to electrically couple the energy source to the end effector; and (c) a plurality of electrodes in electrical communication with the working surface and configured to apply an energy from the energy source to the tissue, where the plurality of electrodes includes a first electrode and a second electrode on opposite sides of one or more intermediate electrodes, where during applying the energy, the plurality of electrodes is configured such that the first electrode delivers a first voltage and the second electrode delivers a second voltage, where the one or more intermediate electrodes each delivers an intermediate voltage, where a voltage potential differential between the first voltage and the second voltage is greater than a voltage potential difference between the first electrode and the intermediate voltage of any of the one or more intermediate electrodes such that tissue is ablated while also limiting thermal variation within the tissue and locations at the tissue surface where surface temperatures exceed a maximum temperature.

In a more detailed embodiment of the first aspect, the end effector comprises a radiofrequency pen. In yet another more detailed embodiment, the end effector comprises a radiofrequency clamp. In a further detailed embodiment, the end effector comprises a radiofrequency pen or pod comprising a vacuum suction. In still a further detailed embodiment, the end effector comprises an expandable device. In a more detailed embodiment, the expandable device comprises an inflatable element. In a more detailed embodiment, the first electrode and the second electrode have a larger width than the intermediate electrode. In another more detailed embodiment, the widths of the first electrode and the second electrode are 2 to 8 mm, the width of the intermediate electrode is smaller than the first electrode and the second electrode, and the total width that includes a maximum voltage potential difference is between 10 and 30 mm. In yet another more detailed embodiment, a length of the plurality of electrodes is configured to create a desired ablation length. In still another more detailed embodiment, the plurality of electrodes comprises three or more electrodes.

In yet another more detailed embodiment of the first aspect, the plurality of electrodes is distributed in a rectangular array. In yet another more detailed embodiment, the first electrode, the second electrode, and the intermediate electrode are distributed annularly, wherein each electrode is concentric with respect to an adjacent electrode. In a further detailed embodiment, a power of each electrode is different than a power of an adjacent electrode. In still a further detailed embodiment, a current of each electrode is different than a current of an adjacent electrode. In a more detailed embodiment, the intermediate electrode comprises a resistance conductor, wherein the resistance conductor is configured to reduce potential differences between the first electrode and the second electrode. In a more detailed embodiment, a voltage potential differential between each adjacent electrode is uniform or non-uniform. In another more detailed embodiment, a maximum voltage potential differential is between 10 volts and 500 volts. In yet another more detailed embodiment, the maximum voltage potential differential is between 30 and 80 volts. In still another more detailed embodiment, a total power output is between 1 watt and 200 watts.

In a more detailed embodiment of the first aspect, the total power output is between 10 and 40 watts. In yet another more detailed embodiment, an applied frequency is between 50 kilohertz and 5,000 kilohertz. In a further detailed embodiment, the applied frequency is between 300 and 500 kilohertz. In still a further detailed embodiment, multiple electrode arrays are situated end-to-end on the tissue ablation device to extend a surface length of an ablated region while maintaining the electrical and a thermal energy in a width and a depth within the ablated region. In a more detailed embodiment, the voltage and current of each electrode is in phase or out of phase with adjacent electrodes, where the phase is a time-dependent phase of applied AC voltage potentials. In a more detailed embodiment, the voltage and current of each of the first electrode and the second electrode is a sinusoidal wave versus time. In another more detailed embodiment, the voltage and current of each of the first electrode and the second electrode is a rectangular wave versus time. In yet another more detailed embodiment, the intermediate electrode is electrically disconnected from the first electrode and the second electrode, where the intermediate electrode transfers current and reduces resistance between the first electrode and the second electrode.

It is a second aspect of the present disclosure to provide a tissue ablation device for ablating tissue, the device comprising: (a) an end effector having a tissue contacting surface; (b) a power source coupled to the end effector; and (c) an array of electrodes in electrical communication with the tissue contacting surface, where each electrode in the array of electrodes is held at a voltage potential, current, or power as provided from the voltage source, where the voltage potential, current, or power of each electrode is different than the voltage potential, current, or power of an adjacent electrode, where the array of electrodes is configured to ablate the tissue while distributing electric potential across a surface of the tissue, such that distribution of the electric potential reduces a temperature variation within the tissue ablated.

It is a third aspect of the present disclosure to provide a method for ablating tissue, the method comprising: (a) positioning an end effector at a target site of the tissue, where the end effector comprises a tissue contacting surface in electrical communication with an array of electrodes; (b) applying voltage potential, current, or power from a power source to each electrode in the array of electrodes, where the array of electrodes comprises a distributed potential and is configured to ablate the tissue while distributing electric potential across the tissue; and (c) ablating the tissue spaced from the end effector while minimizing heat applied to a surface of the tissue in order to generate more uniform and deeper ablations.

It is a fourth aspect of the present disclosure to provide an electrosurgical device, comprising: (a) a first electrode; (b) a second electrode; and (c) an intermediate electrical element, where the first electrode, the second electrode, and the intermediate electrical element are configured to electrically communicate with a target tissue, and where the intermediate electrical element interposes the first electrode and the second electrode.

In a more detailed embodiment of the fourth aspect, the intermediate electrical element comprises at least one intermediate electrode. In yet another more detailed embodiment, the at least one intermediate electrode comprises a plurality of intermediate electrodes. In a further detailed embodiment, the at least one electrical parameter comprises current. In still a further detailed embodiment, the first electrode, the at least one intermediate electrode, and the second electrode are configured to deliver electrical energy to the target tissue, and at least one electrical parameter of the electrical energy varies incrementally between the first electrode, the at least one intermediate electrode, and the second electrode. In a more detailed embodiment, the at least one electrical parameter comprises electrical potential. In a more detailed embodiment, the at least one electrical parameter comprises power.

It is a fifth aspect of the present disclosure to provide an electrosurgical system comprising: (a) an electrosurgical device, comprising: (i) a first electrode; (ii) a second electrode; and (iii) an intermediate electrical element, where the first electrode, the second electrode, and the intermediate electrical element are configured to electrically communicate with a target tissue, and where the intermediate electrical element interposes the first electrode and the second electrode; (b) a resistive voltage divider electrically connected to a first input conductor and a second input conductor, the resistive voltage divider comprising a first resistor and a second resistor, where the first resistor and the second resistor are electrically connected in series between the first input conductor and the second input conductor, and where the first electrode is configured to electrically connect to the first input connector, the second electrode is configured to electrically connect to the second input connector, and the at least one intermediate electrode is configured to electrically connect to at least one intermediate conductor electrically connected between the first resistor and the second resistor.

In a more detailed embodiment of the fifth aspect, the resistive voltage divider is disposed within at least one of a handle, a shaft, an end effector, or a connecting element of the electrosurgical device, the first input conductor and the second input conductor are configured to releasably electrically couple to an electrosurgical generator, the first input conductor is electrically coupled to the first electrode, the second input conductor is electrically coupled to the second electrode, and the at least one intermediate electrode is electrically coupled to the at least one intermediate conductor. In yet another more detailed embodiment, the resistive voltage divider is disposed within an interface component configured to electrically interpose the electrosurgical device and the electrosurgical generator, the first input conductor and the second input conductor are configured to releasably electrically couple to an electrosurgical generator, the first input conductor is configured to releasably electrically couple to the first electrode, the second input conductor is configured to releasably electrically couple to the second electrode, and the at least one intermediate conductor is configured to releasably electrically couple to the at least one intermediate electrode. In a further detailed embodiment, the resistive voltage divider is disposed within the electrosurgical generator, the first input conductor is configured to releasably electrically couple to the first electrode, the second input conductor is configured to releasably electrically couple to the second electrode, and the at least one intermediate conductor is configured to releasably electrically couple to the at least one intermediate electrode.

It is a sixth aspect of the present disclosure to provide an electrosurgical device, comprising: (a) a first electrode; (b) a second electrode; and (c) at least one intermediate electrically resistive element, where the first electrode, the second electrode, and the at least one intermediate electrically resistive element are configured to electrically communicate with a target tissue, and where the at least one intermediate electrically resistive element interposes the first electrode and the second electrode.

In a more detailed embodiment of the sixth aspect, the at least one intermediate electrically resistive element comprises a first electrically resistive element electrically connected to the first electrode and a second electrically resistive element electrically connected to the second electrode, and the first electrically resistive element is not directly electrically connected to the second electrically resistive element. In yet another more detailed embodiment, the first electrically resistive element and the second electrically resistive element are interposed by a gap. In a further detailed embodiment, the gap comprises at least one of an unoccupied space and a non-conductive element. In still a further detailed embodiment, the at least one intermediate electrically resistive element is electrically connected between the first electrode and the second electrode. In a more detailed embodiment, an electrical resistance of the at least one intermediate electrically resistive element is approximately equal to an electrical resistance of a target tissue.

It is a seventh aspect of the present disclosure to provide an electrosurgical device, comprising: (a) a tissue contacting surface in electrical communication with a first electrode, a second electrode, and a plurality of intermediate electrodes; and (b) an electrical input connector, where the first electrode and the second electrode are spaced apart by a first width, where the plurality of intermediate electrodes is disposed sequentially between the first electrode and the second electrode along the first width, and where at least one electrical parameter varies between the first electrode, the plurality of intermediate electrodes, and the second electrode such that the electrical parameter has a first value at the first electrode, a second value at the second electrode, and a respective intermediate value between the first value and the second value at each of the intermediate electrodes.

In yet another more detailed embodiment of the seventh aspect, the intermediate values vary incrementally between the first electrode, each intermediate electrode, and the second electrode.

It is an eighth aspect of the present disclosure to provide an ablation device for creating a lesion in a target tissue, the ablation device comprising: (a) an end effector comprising a tissue engagement portion configured to engage a target tissue, the tissue engagement portion configured for electrical contact with the target tissue and comprising a first tissue contact, a second tissue contact, and an intermediate tissue contact, where the intermediate tissue contact is disposed between the first tissue contact and the second tissue contact, and where the first tissue contact, the intermediate tissue contact, and the second tissue contact are electrically coupled so that, when the end effector is supplied with electrical ablation energy, a magnitude of at least one electrical parameter differs between the first tissue contact, the intermediate tissue contact, and the second tissue contact so that an intermediate tissue contact magnitude is between a first tissue contact magnitude and a second tissue contact magnitude.

In yet another more detailed embodiment of the eighth aspect, the tissue engagement portion comprises a discrete first electrode comprising the first tissue contact and a discrete second electrode comprising the second tissue contact. In yet another more detailed embodiment, the tissue engagement portion comprises a discrete intermediate electrode comprising the intermediate tissue contact. In a further detailed embodiment, the tissue engagement portion comprises a first insulator between the first electrode and the intermediate electrode and a second insulator between the intermediate electrode and the second electrode. In still a further detailed embodiment, the intermediate electrode comprises at least two sequentially disposed, discrete intermediate electrodes, and the magnitude of the at least one electrical parameter differs incrementally between the at least two sequentially disposed, discrete intermediate electrodes. In a more detailed embodiment, the first electrode, the intermediate electrode, and the second electrode are disposed in a line, and the first electrode is disposed as a first outermost electrode at a first end and the second electrode is disposed as a second outermost electrode at a second end. In a more detailed embodiment, the first electrode is nested within the intermediate electrode, and the intermediate electrode is nested within the second electrode. In another more detailed embodiment, the first electrode is nested concentrically within the intermediate electrode, and the intermediate electrode is nested concentrically within the second electrode. In yet another more detailed embodiment, the intermediate electrode and the second electrode comprise nested, concentric, generally stadium-shaped ring electrodes disposed about the first electrode. In still another more detailed embodiment, the first electrode is generally circular, the intermediate electrode is generally semiannular and is disposed about the first electrode, and the second electrode is generally semiannular and is disposed about the intermediate electrode.

In a more detailed embodiment of the eighth aspect, the intermediate electrode and the second electrode are truncated to form a generally bowtie shape. In yet another more detailed embodiment, the first electrode, the intermediate electrode, and the second electrode are truncated to form a generally bowtie shape. In a further detailed embodiment, the tissue engagement portion comprises a semiconductor element comprising the intermediate tissue contact. In still a further detailed embodiment, the semiconductor element has a resistivity greater than a resistivity of the target tissue. In a more detailed embodiment, the semiconductor element further comprises the first tissue contact and the second tissue contact. In a more detailed embodiment, the end effector further comprises a first electrical conductor electrically coupled to the semiconductor element proximate the first tissue contact, the end effector further comprises a second electrical conductor electrically coupled to the semiconductor element proximate the second tissue contact, and the first electrical conductor and the second electrical conductor are configured to receive the electrical ablation energy from an ablation energy source. In another more detailed embodiment, the end effector further comprises an intermediate electrical conductor electrically coupled to the semiconductor element proximate the intermediate tissue contact.

In yet another more detailed embodiment of the eighth aspect, the intermediate electrical conductor is electrically coupled to the first electrical conductor and the second electrical conductor so that, when the first electrical conductor and the second electrical conductor are supplied with the electrical ablation energy, the magnitude of the at least one electrical parameter differs between the first electrical conductor, the intermediate electrical conductor, and the second electrical conductor so that the intermediate tissue contact magnitude is between the first tissue contact magnitude and the second tissue contact magnitude. In yet another more detailed embodiment, the intermediate electrical conductor is electrically coupled to the first electrical conductor by a first resistor, and the intermediate electrical conductor is electrically coupled to the second electrical conductor by a second resistor. In a further detailed embodiment, the tissue engagement portion comprises a discrete first electrode comprising the first tissue contact and a discrete second electrode comprising the second tissue contact. In still a further detailed embodiment, the semiconductor element is electrically coupled to the first electrode and to the second electrode. In a more detailed embodiment, the at least one electrical parameter comprises electrical potential. In a more detailed embodiment, the at least one electrical parameter comprises electrical current. In another more detailed embodiment, the electrical ablation energy comprises radiofrequency electrical energy. In yet another more detailed embodiment, the electrical ablation energy comprises pulsed field ablation electrical energy. In still another more detailed embodiment, the ablation device further includes a shaft disposed proximally on the end effector.

In a more detailed embodiment of the eighth aspect, the ablation device further includes a handle disposed proximally on the shaft. In yet another more detailed embodiment, the ablation device further includes at least one connecting element configured to electrically couple the end effector to an external ablation energy source.

It is a ninth aspect of the present disclosure to provide a method of creating a lesion in a target tissue, the method comprising: (a) positioning a tissue engagement portion of an end effector of an ablation device proximate a target tissue so that a first tissue contact of the tissue engagement portion is in electrical contact with the target tissue, a second tissue contact of the tissue engagement portion is in electrical contact with the target tissue, and an intermediate tissue contact of the tissue engagement portion between the first tissue contact and the second tissue contact is in electrical contact with the target tissue, and (b) creating a lesion in the target tissue by applying electrical ablation energy to the end effector so that a magnitude of at least one electrical parameter or a combination of electrical parameters differs between the first tissue contact, the intermediate tissue contact, and the second tissue contact so that the magnitude at the intermediate tissue contact is less than the magnitude at the first tissue contact and greater than the magnitude at the second tissue contact.

In a more detailed embodiment of the ninth aspect, applying the electrical ablation energy to the end effector comprises applying the electrical ablation energy to a discrete first electrode comprising the first tissue contact and a discrete second electrode comprising the second tissue contact. In yet another more detailed embodiment, applying the electrical ablation energy to the end effector comprises applying the electrical ablation energy to a discrete intermediate electrode comprising the intermediate tissue contact. In a further detailed embodiment, the intermediate electrode comprises at least two sequentially disposed, discrete intermediate electrodes, and applying the electrical ablation energy to the discrete intermediate electrode comprises applying the electrical ablation energy to the at least two sequentially disposed, discrete intermediate electrodes so that the magnitude of the at least one electrical parameter differs incrementally between the at least two sequentially disposed, discrete intermediate electrodes. In still a further detailed embodiment, applying electrical ablation energy to the end effector comprises applying the electrical ablation energy to a semiconductor element comprising the intermediate tissue contact. In a more detailed embodiment, applying electrical ablation energy to the end effector comprises applying the electrical ablation energy to the semiconductor element, the semiconductor element further comprising the first tissue contact and the second tissue contact. In a more detailed embodiment, applying electrical ablation energy to the end effector comprises applying the electrical ablation energy from an ablation energy source to a first electrical conductor and a second electrical conductor, the first electrical conductor is electrically coupled to the semiconductor element proximate the first tissue contact, and the second electrical conductor is electrically coupled to the semiconductor element proximate the second tissue contact. In another more detailed embodiment, applying electrical ablation energy to the end effector comprises applying the electrical ablation energy from the ablation energy source to an intermediate electrical conductor, and the intermediate electrical conductor is electrically coupled to the semiconductor element proximate the intermediate tissue contact.

In yet another more detailed embodiment of the ninth aspect, applying the electrical ablation energy from the ablation energy source to the intermediate electrical conductor comprises applying the electrical ablation energy from the ablation source to the intermediate electrical conductor so that the magnitude of the at least one electrical parameter or a combination of electrical parameters differs between the first electrical conductor, the intermediate electrical conductor, and the second electrical conductor so that the intermediate tissue contact magnitude is between the first tissue contact magnitude and the second tissue contact magnitude. In yet another more detailed embodiment, applying the electrical ablation energy from the ablation source to the intermediate electrical conductor comprises applying the electrical ablation energy from the ablation source to the intermediate electrical conductor from the first electrical conductor via a first resistor and from the second electrical conductor via a second resistor. In a further detailed embodiment, applying electrical ablation energy to the end effector comprises applying the electrical ablation energy to a discrete first electrode comprising the first tissue contact and a discrete second electrode comprising the second tissue contact. In still a further detailed embodiment, the at least one electrical parameter comprises electrical potential. In a more detailed embodiment, the at least one electrical parameter comprises electrical current. In a more detailed embodiment, the electrical ablation energy comprises radiofrequency electrical energy. In another more detailed embodiment, the electrical ablation energy comprises pulsed field ablation electrical energy. In yet another more detailed embodiment, the ablation device comprises a shaft disposed proximally on the end effector, and positioning the tissue engagement portion of the end effector of the ablation device proximate the target tissue comprises positioning the tissue engagement portion of the end effector of the ablation device using the shaft. In still another more detailed embodiment, the ablation device comprises a handle disposed proximally on the shaft, and positioning the tissue engagement portion of the end effector of the ablation device proximate the target tissue comprises positioning the tissue engagement portion of the end effector of the ablation device using the handle. In yet another more detailed embodiment, the ablation device comprises at least one connecting element configured to electrically couple the end effector to an external ablation energy source, and applying the electrical ablation energy to the end effector comprises applying the electrical ablation energy to the end effector via the at least one connecting element.

It is a tenth aspect of the present disclosure to provide a method of ablating tissue, the method comprising: (a) positioning an end effector of an ablation device so that a first contact, a second contact, and an intermediate contact, interposing the first and second contacts, of the end effector physically touches the tissue, where the intermediate contact comprises at least one of an electrode and a semiconductor, where the first contact is in electrical communication with a first electrode, and where the second contact is in electrical communication with a second electrode; and, (b) applying electrical energy to the first and second electrodes so that a magnitude of at least one electrical parameter or a combination of electrical parameters differs between the first contact and the second contact, where the magnitude at the intermediate contact is less than the magnitude at the first contact and greater than the magnitude at the second contact.

It is an eleventh aspect of the present disclosure to provide an ablation device for ablating tissue, the ablation device comprising: (a) an end effector comprising a first contact, a second contact, and an intermediate contact interposing the first and second contacts, the intermediate contact comprising a plurality of intermediate electrodes, the first contact in electrical communication with a first electrode, the second contact in electrical communication with a second electrode, where the first electrode and the second electrode are spaced apart from one another a first distance, where the first electrode and the intermediate contact are spaced apart from one another a second distance, where the second electrode and the intermediate contact are spaced apart from one another a third distance, where the first distance is greater than either the second distance or the third distance, and where a surface area of at least one of the first and second electrodes is a multiple of a surface area of any one of the plurality of intermediate electrodes, where when the end effector contacts the tissue, the first contact, the intermediate contact, and the second contact are electrically coupled, where when the first electrode and the second electrode are supplied with electrical ablation energy, a magnitude of at least one electrical parameter differs between the first contact, the intermediate contact, and the second contact so that the magnitude at the intermediate contact is less than the magnitude at the first contact and greater than the magnitude at the second contact. The geometry of the first, second, and additional electrodes may be uniform between themselves in width and length, or vary in width and length, with the intent to tailor the applied power density as a function of location or other characteristic such as applied pressure.

It is an aspect of the present disclosure to provide any method, process, apparatus, or system comprising one or more elements described herein. It is as aspect of the present disclosure to provide any combination of any one or more elements described herein.

Other aspects, features, and embodiments of the present disclosure will become apparent to those of ordinary skill in the art, upon reviewing the following description of specific, example embodiments of the present disclosure in conjunction with the accompanying figures. While features of the present disclosure can be discussed relative to certain embodiments and figures below, all embodiments of the present disclosure can include one or more of the advantageous features discussed herein. In other words, while one or more embodiments can be discussed as having certain advantageous features, one or more of such features can also be used in accordance with the various embodiments of the disclosure discussed herein. In similar fashion, while example embodiments can be discussed below as device, system, or method embodiments, it should be understood that such exemplary embodiments can be implemented in various devices, systems, and methods.

### BRIEF DESCRIPTION OF THE DRAWINGS

The present disclosure is best understood from the following detailed description when read with the accompanying figures.

FIG. 1A illustrates a perspective view of an ablation device according to one or more variations of the present disclosure.

FIG. 1B illustrates a side view of one variation of an ablation device having a radiofrequency pen.

FIG. 1C illustrates a side view of one variation of an ablation device having a radiofrequency pen applied to tissue.

FIG. 1D illustrates a side view of one variation of an end effector having a rigid backing.

FIG. 1E illustrates a side view of one variation of an end effector having a flexible backing.

FIG. 1F illustrates a side view of one variation of an end effector having multiple stacked arrays of electrodes.

FIG. 1G illustrates a bottom view of one variation of an end effector having multiple stacked arrays of electrodes.

FIG. 2A illustrates a side view of one variation of an ablation device having a radiofrequency pen and a suction line.

FIG. 2B illustrates a front view of one variation of an ablation device having a flexible backing and a vacuum line.

FIG. 2C illustrates a front view of yet another variation of an ablation device having a flexible backing and a vacuum line.

FIG. 2D illustrates a front view of the ablation device of FIG. 2B being applied to tissue.

FIG. 2E illustrates a front view of the ablation device of FIG. 2C being applied to tissue.

FIG. 3A illustrates a perspective view of an ablation device coupled to a generator and having an ablation clamp.

FIG. 3B illustrates a side view of one variation of an ablation device having an ablation clamp.

FIG. 4 illustrates a side view of one variation of an ablation device having an expandable member.

FIG. 5 illustrates a bottom view of one variation of an ablation device having concentric electrodes.

FIG. 6 illustrates a bottom view of one variation of an ablation device having semiconductor electrodes in a center of an electrode array.

FIG. 7 illustrates a perspective view of an ablation device coupled with various electrical devices.

FIG. 8 illustrates a simplified schematic view of an example electrosurgical system including an example resistive voltage divider.

FIG. 9 illustrates a simplified bottom view of an example electrode arrangement with annotations showing example maximum voltages.

FIG. 10 illustrates a simplified bottom view of an example electrode arrangement with annotations showing example dimensions.

FIG. 11 is a simplified bottom view of an example electrode arrangement with annotations showing example dimensions.

FIG. 12 is a simplified bottom view of an example electrode arrangement with annotations showing example dimensions.

FIG. 13A is a simplified bottom view of an example electrode arrangement with annotations showing example dimensions.

FIG. 13B is a simplified bottom view of an example electrode arrangement with annotations showing example dimensions.

FIG. 14 is a simplified bottom view of an example electrode arrangement disposed on a substrate.

FIG. 15 is a simplified bottom view of an example electrode arrangement with annotations showing example dimensions.

FIG. 16 is a simplified bottom view of an example electrode arrangement with annotations showing example dimensions.

FIG. 17 is a simplified bottom view of an example electrode arrangement with annotations showing example dimensions.

FIG. 18 is a simplified bottom view of an example concentric electrode arrangement.

FIG. 19 illustrates a cross-section view of an example dome-shaped end effector with interior electrodes.

FIG. 20 illustrates a bottom view of an example tiled electrode arrangement.

FIG. 21 illustrates a cross-section view of an example electrosurgical device configured for closed loop active cooling.

FIG. 22 illustrates a cross-section view of an example electrosurgical device configured for passive cooling.

FIG. 23 illustrates a cross-section view of an example end effector including an expandable member in the form of an inflatable element.

FIG. 24 illustrates a perspective view of an example electrode arrangement disposed on a substrate.

FIG. 25 illustrates a perspective view of an example electrode arrangement.

FIG. 26 illustrates a perspective view of an example end effector including an electrode arrangement.

FIG. 27 illustrates a perspective view of an example end effector including an electrode arrangement.

FIG. 28 illustrates a perspective view of an example end effector including an electrode arrangement.

FIG. 29 illustrates a cross-sectional view of an example current density in a target tissue caused by a two-electrode, bipolar ablation device.

FIG. 30 illustrates a cross-sectional view of an example current density in a target tissue caused by an example ablation device including a first electrode, a second electrode, and four intermediate electrodes.

FIG. 31 illustrates a cross-sectional view of an example voltage potential in a target tissue caused by an example ablation device including an electrode arrangement generally similar to that shown in FIG. 10.

FIG. 32 illustrates a cross-sectional view of example temperature in the target tissue caused by an example ablation device including an electrode arrangement generally similar to that shown in FIG. 10.

FIG. 33A is a simplified bottom view of an example electrode arrangement including generally rectangular electrodes.

FIG. 33B is a simplified bottom view of an example electrode arrangement including generally rectangular electrodes.

FIG. 33C is a simplified bottom view of an example electrode arrangement including generally rectangular electrodes.

FIG. 34A is a simplified bottom view of an example electrode arrangement including nested, generally circular and/or annular ring electrodes.

FIG. 34B is a simplified bottom view of an example electrode arrangement including nested, generally circular and/or annular ring electrodes.

FIG. 34C is a simplified bottom view of an example electrode arrangement including nested, generally circular and/or annular ring electrodes.

FIG. 35A is a simplified bottom view of an example electrode arrangement including nested, generally elliptical ring electrodes.

FIG. 35B is a simplified bottom view of an example electrode arrangement including nested, generally elliptical ring electrodes.

FIG. 35C is a simplified bottom view of an example electrode arrangement including nested, generally elliptical ring electrodes.

FIG. 35D is a simplified bottom view of an example electrode arrangement including nested, generally elliptical ring electrodes.

FIG. 36A is a simplified bottom view of an example electrode arrangement including nested, generally stadium-shaped ring electrodes.

FIG. 36B is a simplified bottom view of an example electrode arrangement including nested, generally stadium-shaped ring electrodes.

FIG. 36C is a simplified bottom view of an example electrode arrangement including nested, generally stadium-shaped ring electrodes.

FIG. 36D is a simplified bottom view of an example electrode arrangement including nested, generally stadium-shaped ring electrodes.

FIG. 36E is a simplified bottom view of an example electrode arrangement including nested, generally stadium-shaped ring electrodes.

FIG. 36F is a simplified bottom view of an example electrode arrangement including nested, generally stadium-shaped ring electrodes.

FIG. 36G is a simplified bottom view of an example electrode arrangement including nested, generally stadium-shaped ring electrodes.

FIG. 36H is a simplified bottom view of an example electrode arrangement including nested, generally stadium-shaped ring electrodes.

FIG. 37A is a simplified bottom view of an example electrode arrangement including truncated nested, generally circular and/or annular ring electrodes.

FIG. 37B is a simplified bottom view of an example electrode arrangement including truncated nested, generally circular and/or annular ring electrodes.

FIG. 37C is a simplified bottom view of an example electrode arrangement including truncated nested, generally circular and/or annular ring electrodes.

FIG. 37D is a simplified bottom view of an example electrode arrangement including truncated nested, generally circular and/or annular ring electrodes.

FIG. 38A is a bottom view of an example electrode arrangement including a semiconductor electrode interposing two outer electrodes.

FIG. 38B is a simplified elevation view of the embodiment of FIG. 38A.

FIG. 39 is a is a simplified elevation view of an example electrode arrangement including a semiconductor electrode comprising a semiconductor layer disposed on metal conductors.

FIG. 40 is a simplified elevation view of an example electrode arrangement including a semiconductor electrode with conductors embedded therein.

FIG. 41 is a simplified bottom view illustrating a comparison of symmetric and asymmetric electrode arrangements.

FIG. 42 is a simplified bottom view illustrating dimensions of a rectangular electrode array which may be varied.

FIG. 43 is a simplified bottom view illustrating dimensions of a concentric electrode array which may be varied.

### DETAILED DESCRIPTION

The detailed description set forth below, in connection with the appended drawings, is intended as a description of various configurations and is not intended to represent the only configurations in which the aspects described herein can be practiced. The detailed description includes specific details for the purpose of providing a thorough understanding of the various aspects. However, it will be apparent to those skilled in the art that these concepts can be practiced without these specific details. In some instances, well-known structures and components are shown in block diagram form in order to avoid obscuring such aspects.

All examples and illustrative references are non-limiting and should not be used to limit the claims to specific implementations and embodiments described herein and their equivalents. For simplicity, reference numbers can be repeated between various examples. This repetition is for clarity only and does not dictate a relationship between the respective embodiments. Finally, in view of this disclosure, particular features described in relation to one aspect or embodiment can be applied to other disclosed aspects or embodiments of the disclosure, even though not specifically shown in the drawings or described in the text.

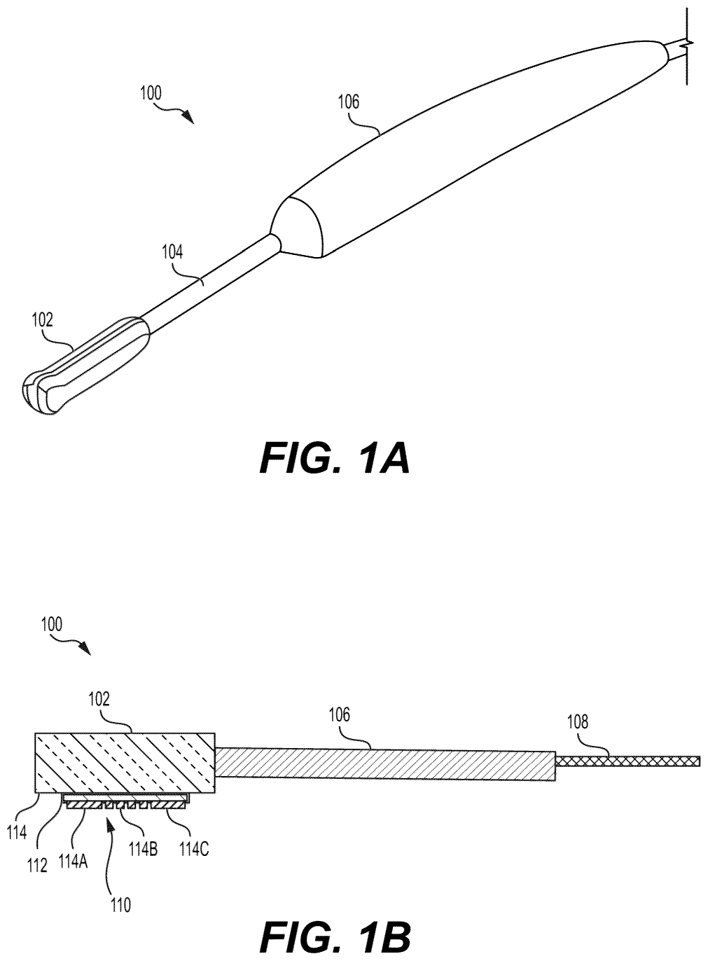

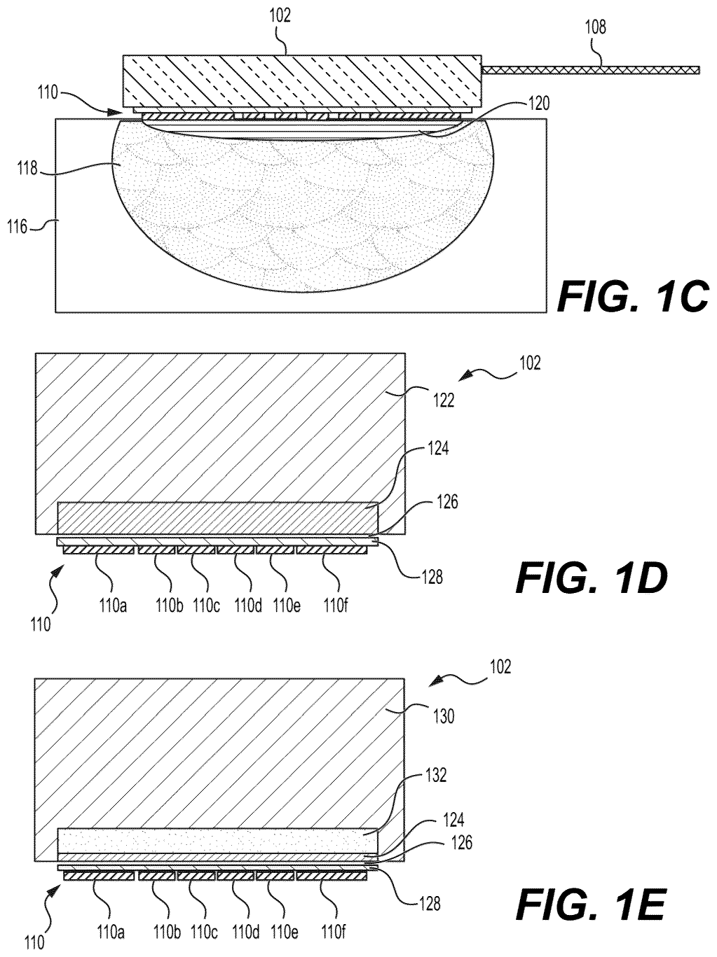

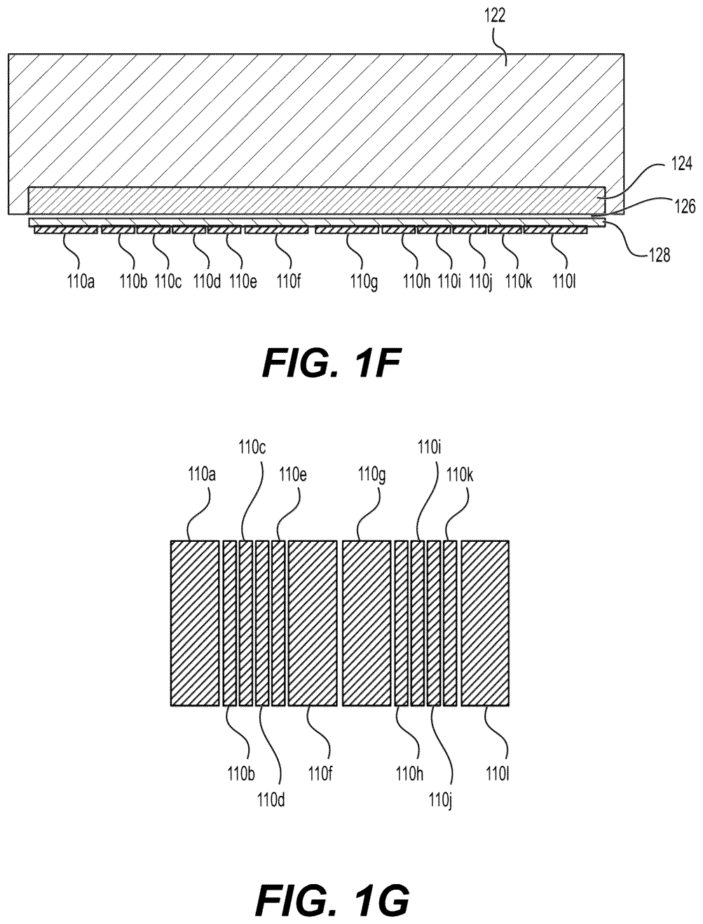

The present disclosure includes various electrosurgical devices, including ablation devices. FIG. 1A shows an example ablation device 100, which provides context for various alternative embodiments and optional features described herein. Unless otherwise indicated, any component, feature, method, etc. described herein may be utilized, alone or in any combination, in connection with an ablation device generally similar to ablation device 100. Ablation device 100 can have an end effector 102 connected to a distal end of a shaft 104 and a handle 106 connected to a proximal end of the shaft 14. The shaft 14 can be straight and substantially rigid. However, flexible, curved, malleable, articulated, or other shafts could also be used depending on a variety of considerations.

As seen in FIG. 1B, a connecting element, such as a cable 108, can be coupled to the handle 106 for connecting to devices such as power sources (e.g., external ablation energy sources). The end effector 102 can extend along a longitudinal axis of the device 100 and can have a total width of about 26 mm. The end effector 102 can include a tissue engagement portion or working surface 114. As used herein, “working surface” may refer to a surface that is configured to come into contact with a target tissue. A working surface may include one or more individual surfaces, which may be contiguous or separated, and may include surfaces that are in any shape (planar, curved, concave, convex, etc.). The working surface 114 can comprise one or more insulators 112 (or insulator portions) and one or more electrodes 110, which can be capable of being energized with electrical ablation energy, such as with bipolar RF energy or pulsed field ablation energy. As used herein, “electrode” may refer to an element configured to deliver electrical energy to a target tissue through contact with the target tissue. Each electrode 110 can include a smooth surface area for contacting tissue. A variety of different metals or low electrical resistance materials can be used for the electrodes that are sufficiently electrically conductive to transfer potential and current to the tissue via ionic current density. For example, the electrodes 110 can be made of copper, nickel, gold, stainless steel, platinum, platinum-iridium, titanium, tin, metal on Kapton, polymer-metal composites, hydrogels, or combinations thereof. The tissue engagement portion 114 may include a plurality of tissue-contacting positions, such as a first tissue contacting position 114A, an intermediate tissue-contacting position 114B, and a third tissue-contacting position 114C. The intermediate tissue-contacting position 114B may be disposed between the first tissue-contacting position 114A and the third tissue-contacting position 114C. In the illustrated embodiment, the electrodes 110 may form the tissue contacting positions 114A, 114B, 114C.

As seen in FIG. 1C, a stepped electrodes configuration can result in application of the energy to a tissue while ablating the tissue in order to limit temperature variation and extreme temperatures in the tissue (e.g., at tissue region 120). As a result, the surface heating of the tissue can be reduced while still achieving proper ablation deep within the tissue ablated region 118 (e.g., the lesion), at temperatures of about 55° C., for example. In some cases, the depth of ablation after 80 seconds can be about 10 mm from the surface of the tissue. Electrodes can be controlled independently to generate a consistent ablation over a desired width while adjusting for different thicknesses or impedances of tissue. Power outputs can be adjusted based on measured impedance and/or temperature of the tissue.

In other variations, one or more of the electrodes 110, such as one or more of the multiple intermediate electrodes, can be replaced with a moderate electrical resistance material, such as a semiconducting material having an electrical conductivity of about 0.1 Siemens per meter (S/m) to 100 S/m. This moderate resistance, semiconducting material is used to extend the electrodes towards the center, thereby reducing the applied voltage and current in this center region by means of the reduced electrical conductivity of the material. As used herein, “semiconducting material” may refer to a material with electrical properties intermediate between a good conductor and a good insulator.

FIG. 1D shows a cross-sectional view of one variation of a rigid end effector 102. In this variation, the end effector 102 is generally rigid such that the electrodes 110 cannot substantially flex. In the illustrated embodiment, the electrodes 110 are discrete. As used herein, “discrete” may refer to an electrode that presents a tissue-contacting surface that is distinct from other nearby components, such as other electrodes. For example, the end effector 102 can comprise a plastic insulator 122 for backing and which interposes the discrete electrodes 110. The plastic insulator 122 can be about 1-10 mm thick. The end effector 102 can further comprise a thermal conductor 124 within the plastic insulator 122 to help spread heat and minimum hot spots, although this is not critical for overall function. The thermal conductor 124 can be between 0.01-10 mm thick. A polyimide film or other electrical insulator 128 having about 0.1 mm thickness can be attached to the thermal conductor 124 via an adhesive or thermal bonding process 126. In exemplary form, the thermal conductor may be a metal or metal alloy including, without limitation, aluminum and copper.

The plurality of electrodes 110 can be placed on the polyimide film or other electrical insulator 128 and spaced along the width of the end effector 102. The electrodes 110 can each have a width of about 0.1 mm to about 100 mm, a thickness of about 0.01 to 10 mm, and a length of about 20 mm. The electrodes 110 can be spaced apart by gaps about 0.5 mm in width. In the variation shown in FIG. 1D, the outer electrodes 110 can have widths of about 4 mm and the intermediate electrodes can have widths of about 1.5 mm. The electrode widths and configurations can vary as necessary to reduce current density, reducing surface heating and increasing a width of ablation. For example, in some embodiments, it may be advantageous to utilize relatively thin electrodes to reduce electrode mass and/or to reduce heat flux out of the electrodes. Further, multiple metal electrodes or higher-resistance (e.g., semiconducting) electrodes adjacent to metal electrodes can be used in other configurations to reduce the potential on the upper tissue surface. A total active width of the electrode region (e.g., from one outer electrode to the other) can be about 22.5 mm. Alternatively, or in combination, the electrodes 110 can be arrayed and stepped in a direction perpendicular to the longitudinal axis of the end effector to locally adjust electrical output to selectively ablate regions of the heart, where the electrical energy applied to these regions can be controlled actively or passively.

The electrodes 110 can have sharp corners or rounded corners. Sharp corners can lead to a higher local current density when heat is applied to the tissue. Optionally, the device 100 can comprise an active cooling mechanism for cooling the heart tissue and/or device 100. The device can also optionally comprise a second electrically insulating layer to cover any electrical leads within the electrodes 110.

Electrodes 110a, 110b, 110c, 110d, 110e, and 110f can be distributed in an array. The plurality of electrodes can include a first electrode 110a and a second electrode 110f on opposite sides of one or more intermediate electrodes 110b-110e. During energy application, the plurality of electrodes can be configured such that there is a voltage differential between the first electrode 110a and second electrode 110f. The one or more sequentially disposed intermediate electrodes 110b-110e can deliver a smaller, incremental voltage differential relative to the outer electrodes. The electrodes 110 can be bipolar and/or multi-polar electrodes. The electrodes 110 can be oriented perpendicular to a longitudinal axis of the end effector 102 or can be parallel to a longitudinal axis of the end effector 102 or offset at angles between zero and ninety degrees.

The voltage potential differential between the first voltage and the second voltage can be greater than a voltage potential differential between the first electrode and the intermediate voltage of any of the one or more intermediate electrodes. The electrodes 110 can also range between 0 V and 75 V, at intervals of 15 V between adjacent electrodes. For example, electrode 110a can have a voltage of 75 V, electrode 110c can have a voltage of 45 V, electrode 110d can have a voltage of 30 V, electrode 110e can have a voltage of 15 V, and electrode 110f can have a voltage of 0 V. Electrodes 110 can also range between 0 V and 50 V, at intervals of 10 V between adjacent electrodes. In other variations, the maximum voltage potential differential can be between 10 V and 500 V with varying intervals between electrodes 110. In other variations, voltage intervals may not be constant between each electrode. In other variations, other electrical parameters such as electrical current or power may be stepped between electrodes instead of voltage.

The voltage potential differential between electrodes 110 and resulting gradient of the voltage of the end effector can be achieved. The voltage can be provided through the circuitry and/or resistors of the ablation device 100 which modify the potential applied to each electrode as originally sourced from a power supply connected to the ablation device 100 (for example, an ablation sensing unit (ASU) generator 308 as further described herein). The voltage may also be provided through independent power supplies, or combinations thereof.

The stepped electrodes limit the potential difference and, therefore, the current density in the upper surface of the tissue while maintaining current density deep in the tissue, which can lead to safer, more efficient ablations. The voltage applied by the electrodes 110 can also be cycled on/off in intervals to balance ablation depth and surface temperature as needed.

FIG. 1E shows a cross-sectional view of one variation of a flexible end effector 102. A silicone insulator 130 can be utilized for backing to provide flexibility for the end effector 102. The silicone insulator 130 can be about 1-10 mm thick. The insulator can alternatively be made of filled polyurethane or other elastomer, standard plastics, glass-filled or carbon-filled polymers, polyimide over aluminum, various metals with a sealed vacuum interior, or combinations thereof. A filled silicone or other elastomer thermal conductor 132 can be provided towards the working surface 114 of the end effector 102, although this is not critical for overall function. The filled elastomer thermal conductor 132 can be about 0.1-10 mm thick. The thermal conductor 132 can alternatively be made of foamed polyurethane or other elastomer. Both the silicone insulator 130 and filled silicone thermal conductor 132 can be flexible and can provide the end effector 102 with the ability to flex against the heart tissue and conform to the curved surface of the heart, providing more tissue contact area for ablation. The end effector 102 can further comprise a metal thermal conductor 124 within the silicone insulator 130, although this is not critical for overall function. The metal thermal conductor 124 can be about 0.01-10 mm thick. A polyimide film 128 having about 0.1 mm thickness can be attached to an aluminum thermal conductor 124 via an adhesive or thermal bonding process 126. Overall thermal efficiency and mechanical flexibility can be controlled by changing layer thicknesses and material moduli of the components of the end effector 102.

FIG. 1F shows a cross-sectional view of a variation of the end effector 102 having multiple sets of electrodes 110 (e.g., two sets of six electrodes). Multiple stacked rectangular electrode arrays can be situated end-to-end on the tissue ablation device 100 to extend a surface length of an ablated region while maintaining the electrical and a thermal energy in a width and a depth within the ablated region (e.g., three or more sets). The electrodes 110 can be positioned along the longitudinal axis of end effector 102. Each set of electrodes 110 can have adjacent electrodes ranging between 0 V and 50 V, at intervals of 10 V between adjacent electrodes. The electrodes 110 can also range between 0 V and 75 V, at intervals of 15 V between adjacent electrodes. The total active region length can be around 45 mm, but can depend on the total amount of sets of electrodes 110. FIG. 1G shows a bottom view of two sets of electrodes 110 positioned end-to-end on the end effector 102. In some variations, the active width of the electrode region (e.g., from one outer electrode the other) can be less than about 12 mm to fit through trocars used for minimally invasive surgical procedures.

Alternatively or in combination, the contact layer of electrodes 110 can be roughened and then coated with a thin polymeric film with high ionic conductivity (e.g., ionic-doped hydrogel) to reduce impedance between tissue and metal, increase capacitance, and thus to reduce surface heating. Additionally, a heat sink behind the electrodes 110 can assist in lowering surface temperature as well as keeping an upper surface of the end effector 102 cool. The heat sink can vary in thickness depending on various factors (e.g., the trocar diameter). The hydrogel can also be used to reduce adhesion between the tissue and the electrodes 110. Tissue current can increase with increased capacitance as well with decreased interfacial impedance between electrode and tissue. Interfacial impedance generally decreases with increasing electrode surface area. As tissue is ablated and as moisture is removed, interfacial impedance and tissue conductivity decreases.

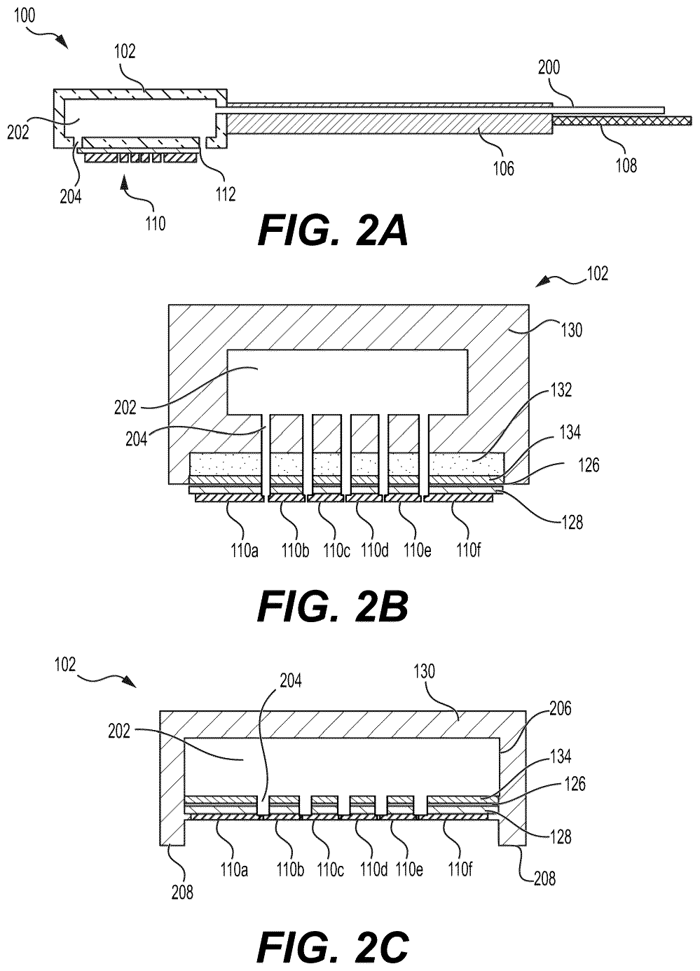

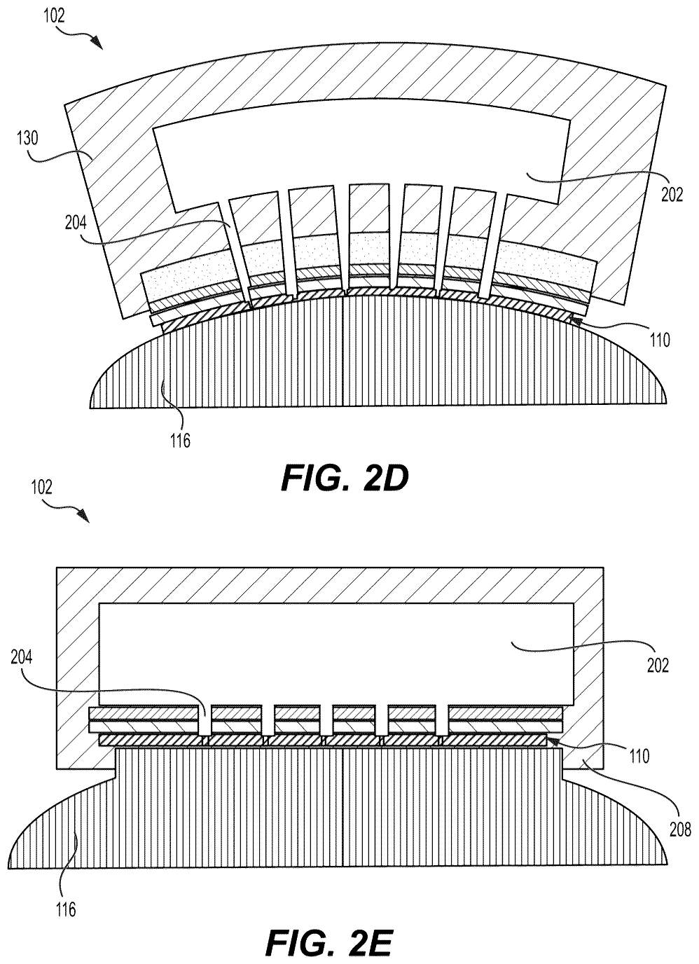

FIG. 2A shows an ablation device 100 having an end effector 102 comprising a suction line 200 along the longitudinal axis of the device 100. The suction line 200 can be provided either disposed within shaft 104 or along an outside of shaft 104 (see FIG. 1). The suction line 200 can extend to an opening 202 within the end effector 102. The suction line 200 can provide a vacuum for adherence to the tissue without applying unnecessary external force to the tissue. The vacuum can ensure a more efficient ablation upon connection of the electrodes 110. Efficient ablations can be measured via time to reach transmurality, required power, uniformity of heating, depth of ablation, lower backside temperatures, less tissue surface damage, or combinations thereof.

As seen in FIG. 2B, end effector 102 can have some of the properties of the flexible end effector 102 of FIG. 1E, but with an opening 202 at the distal end of suction line 200. The opening 202 can be made with an open-cell foam layer to support the overall structure and reduce backside heating, molded in gaps, molded in connectors to connect to the opening 202, or combinations thereof. Gaps 204 can be made within the electrodes 110, insulating film 128, adhesive 126, thermal conductor 132, and thermal conductor 134 to connect the opening to outer surfaces of the electrodes 110.

FIG. 2C shows another variation of an end effector 102 comprising a suction line 200 and having a rigid shell 206 along the silicone insulator 130. The silicone insulator 130 can have a thickness of about 6 mm or less. The electrodes 110 can be fabricated separately from the opening 202 within the silicone insulator 130, allowing for easier fabrication. The rigid shell 206 can provide more rigidity to the end effector 102 when the flexible edges 208 partially extend past the electrodes 110.

As seen in FIG. 2D, the end effector 102 can be placed next to the target tissue, such as heart tissue 116. The end effector 102 can then be activated by the suction line 200 providing a vacuum to the opening 202. The vacuum can travel through the gaps 204 through the electrodes 110 to provide a vacuum to pull the tissue 116 between the electrodes 110 for consistent electrical connection with the electrodes 110. The electrodes 110 can thus be oriented across tissues, allowing current to go deeper into the tissue. When desired, the end effector 102 can flex along its longitudinal axis or perpendicular to its longitudinal axis or alternatives therebetween.

Alternatively, as seen in FIG. 2E, tissue can be pulled into the end effector 102 via the suction line 200 (see FIG. 2A). Upon pulling of tissue against or at least partially into the end effector 102, the silicone insulator 130 of the end effector 102 can have flexible edges 208 that partially extend past the electrodes 110, allowing the end effector 102 to cup and seal against the heart tissue. As vacuum is being applied, the heart tissue can be pulled against electrodes 110, increasing ablation efficiency by improving contact between electrodes 110 and the heart tissue, particularly in minimally invasive procedures.

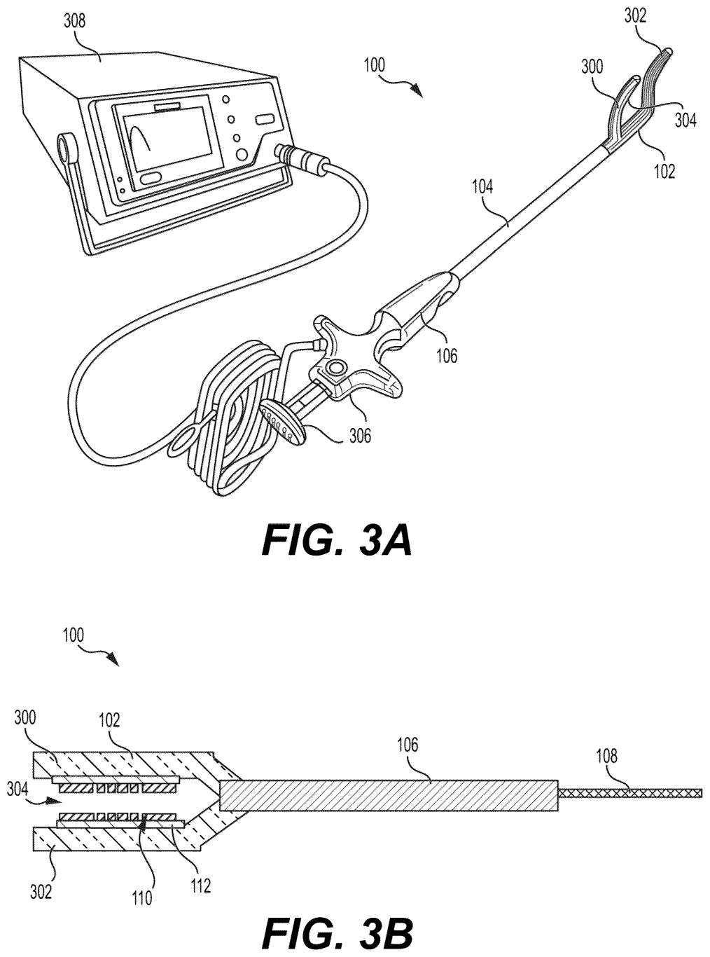

FIG. 3A shows an ablation device 100 having an end effector 102 connected to a distal end of a shaft 104 and a handle 106 connected to a proximal end of the shaft. In this variation, the end effector 102 can be a surgical ablation clamp such that RF energy flows between the two sides of a bipolar clamp. The surgical ablation clamp can provide increased electrical contact with the tissue and increased RF energy from both sides of the tissue, which can lead to improved heating through the tissue thickness 102. The ablation clamp can reduce free liquid that could cause steam pops during the procedure.

The end effector 102 can have a proximal jaw 300 and a distal jaw 302. The proximal and distal jaws 300, 302 are shown spaced apart for the reception of tissue therebetween, but at least one of the proximal and distal jaws 300, 302 can be movable to clamp tissue therebetween. To this end, proximal and distal jaws 300, 302 may be operably coupled to a closure trigger 306 extending proximally from the handle 106 such that it is operable with one hand so that distal movement of closure trigger 306 brings the proximal and distal jaws 300, 302 together. Likewise, proximal movement of closure trigger 306 moves the proximal and distal jaws 300, 302 apart. The proximal and distal jaws 300, 302 are shown extending at an angle from the shaft 104, but can be at any angle with the shaft 104. Electrodes 110 can be placed along the jaws 300, 302 and can apply energy to opposite sides of the tissue to flow energy through the thickness of the tissue, forming transmural ablations. The electrodes 110 can each have a width of about 0.3 mm, a height of about 0.7 mm, and a length of about 63.5 mm.

As seen in the cross-sectional view of the end effector 102 in FIG. 3B, the electrodes 110 can be disposed on working surfaces of jaws 300, 302. The electrodes 110 can be configured in any configuration as previously described or as hereafter described. The jaws 300, 302 can be used to clamp tissue 116 before energy is applied to electrodes 110. Energy can be applied via an ASU generator 308, which will be further described herein.

The clamping pressure can press the jaws 300, 302 into the tissue to create a gap 304 that is typically less than the tissue thickness to be ablated.

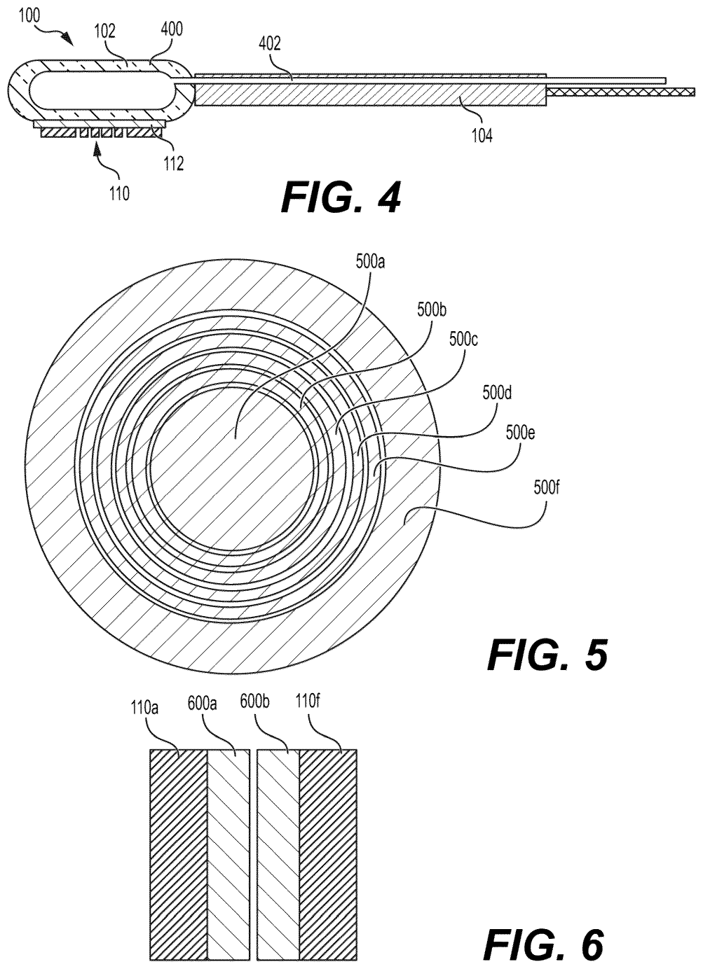

As seen in FIG. 4, the end effector 102 can include an expandable member 400. An air or gas channel 402 can be connected to the expandable member 400 to selectively deflate and inflate the expandable member 400. The air channel 402 can be provided either disposed within shaft 104 or along an outside of shaft 104. Alternatively, or in combination, the expandable member 400 can also be actuated within an actuation cable disposed within shaft 104 or along an outside of shaft 104. The expandable member 400 can be a balloon.

FIG. 5 shows an electrode configuration in a nested, concentric array. As used herein, “nested” may refer to an arrangement in which one or more electrodes are disposed generally within one or more other electrodes, such as an inner electrode being partially or totally circumscribed by an outer electrode. As used herein, “concentric” may refer to an arrangement in which one or more electrodes are disposed around a common center point or shape. Some example electrode arrays may be nested, concentric, or both nested and concentric. The electrodes 110 can be in the form of active electrodes as a circular electrode 500a and rings 500b-f, wherein each electrode of the array of electrodes is concentric with respect to an adjacent electrode. The circular electrode 500a and rings 500b-f can have stepped voltage potential differentials between adjacent electrodes. For example, the voltage potential differential can differ from an outermost ring 500f to an innermost electrode 500a at a uniform or non-uniform intervals. As such, the stepped voltage potential differential of the electrodes distributed annularly can result in minimizing surface heating at the tissue and providing deep ablations at the heart tissue.

FIG. 6 shows a device 100 with one or more inner electrodes or resistance conductors (e.g., semiconductor electrodes) 600a, 600b at a center of the array of electrodes 110. The one or more resistance conductors (e.g., semiconductor electrodes) can be configured to reduce potential differences across the electrodes and can be composed of an intermediate resistance material (e.g., about 0.01 S/m to 1000 S/m). The resistance conductors 600a, 600b can be disconnected from the external circuits and can transfer current and reduce resistance between the outermost electrodes 100a, 100f, thereby reducing heating and temperature increases at the tissue surface. In some example embodiments, resistance conductors and/or semiconductor elements may have a resistivity that is greater than a resistivity of the target tissue. In some example embodiments, resistance conductors and/or semiconductor elements may have a resistivity that is about the same as a resistivity of the target tissue.

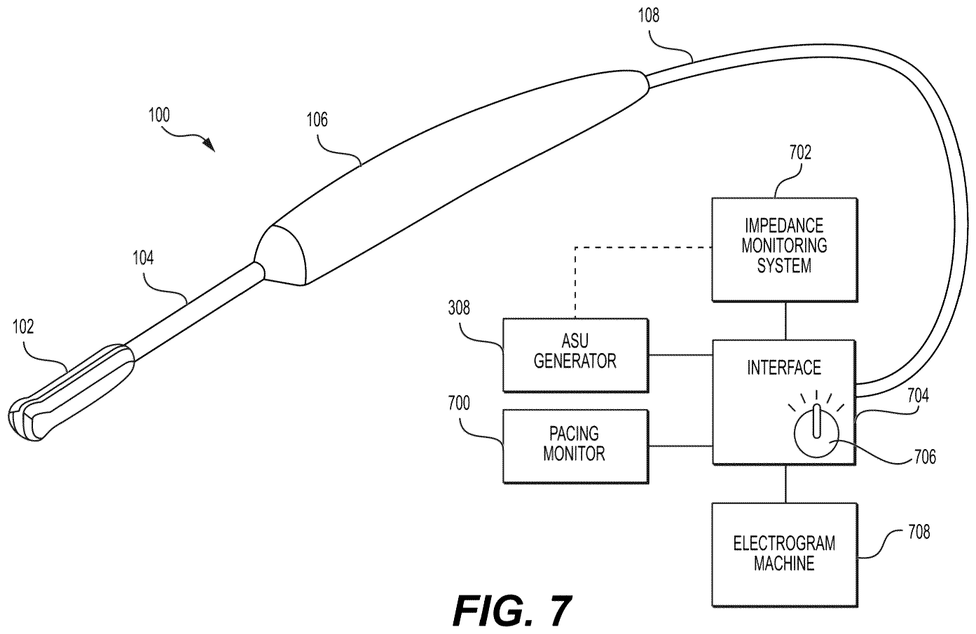

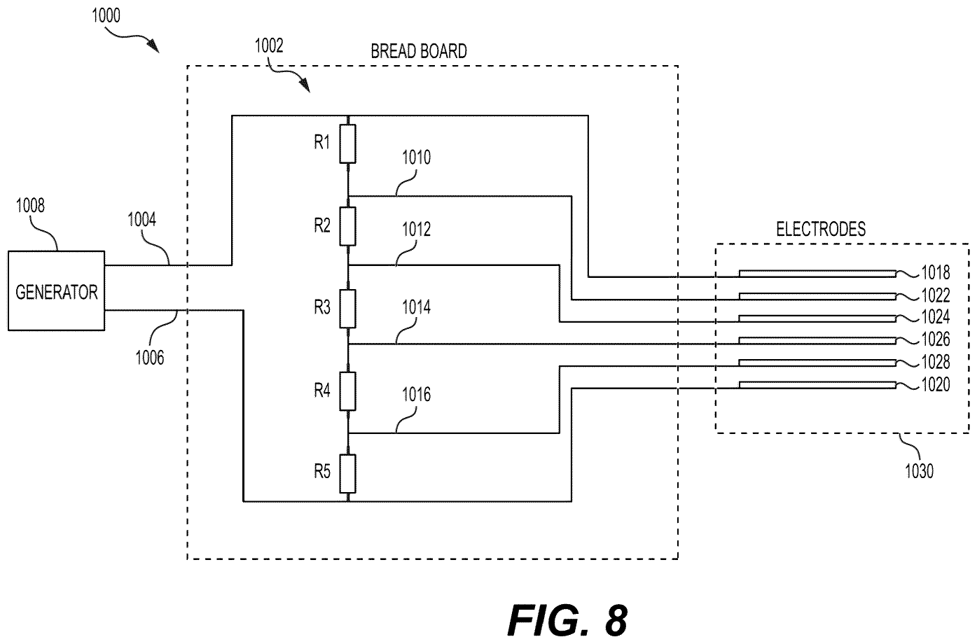

FIG. 7 shows the ablation device 100 having an electrical cable 108 coupled thereto. The electrical cable 108 can extend to a power source at its proximal end. During ablation, the power source can sense and measure tissue properties such as impedance across the electrodes 110 (see e.g., FIG. 4) as tissue is ablated and can change electrical parameters such as power, current, and voltage. The ablation device 100 can be combined with a cable 108 that operably couples the ablation device 100 to a number of different common operating room equipment, devices, and/or sensors that can include the ASU generator 308 or similar generator to create lesions with electrodes 110 (FIG. 1B), a pacing monitor 700 to provide electrical stimulus to tissue, an impedance monitoring system 702 for measuring tissue impedance and an electrogram machine 708 for measuring at least one of voltage, electrical conduction, conduction time, conduction velocity, and signal phase angle of the electrical signals that cause the heart to beat. Thus, the cable 108 in combination with an electrosurgical device such as ablation device 100 can provide the surgeon with a single or multiple electrode device that could be used in lieu of a number of preexisting electrode handheld devices for use in surgery. A switch 706 can be added to the interconnector 704 to operably connect or disconnect one or more of the interconnected devices from the electrode(s) of a surgical device. Additionally, other electrical circuitry or components may be incorporated into the interconnector 704 such as diodes or switching circuitry. The circuitry may protect interconnected sensing equipment from ablative energies or provide real-time controls or switching circuitry. Thus, a surgeon may actuate the ASU generator 308 with a foot pedal and the interconnector 704 would engage and protect sensitive pieces of equipment like an electrogram machine 708. For ablation device 100, each of the interconnected devices can be operably connected or disconnected to the first pole electrode and/or the second pole electrode, or any other electrodes. If additional electrodes are present on a surgical device that can connect to the interconnector 704, the interconnector 704 may accommodate the additional electrodes. The electrodes may be connected in any combination that meets the needs of an energy delivery device or a sensing device, and this may be accomplished at the interconnector 704. A power and/or current of each electrode can be different than a power and/or current of an adjacent electrode.

A total power output of the device 100 can be, for example, between 1 watt and 200 watts. An applied frequency of the device can be, for example, between 50 kilohertz and 5,000 kilohertz. The voltage and current of each electrode can be in phase or out of phase with adjacent electrodes. The phase can be a time-dependent phase of applied voltage potentials. The voltage and current of each electrode can be a sinusoidal wave versus time or a rectangular wave versus time. Power can be applied in multiple time steps to take advantage of thermal conduction through the tissue to heat deep in the tissue without overheating the tissue surface.

FIG. 8 illustrates a simplified schematic view of an example electrosurgical system 1000 including an example resistive voltage divider 1002, according to at least some aspects of the present disclosure. The electrosurgical system 1000 may be generally similar in structure and operation to other electrosurgical systems and related components described herein, and repeated description of similar structures and operations is omitted for brevity. In the illustrated embodiment, the voltage divider includes a plurality of series-connected resistive elements (e.g., resistors R1, R2, R3, R4, and R5), which are electrically connected between a first input conductor 1004 and a second input conductor 1006.

In the illustrated embodiment, the first input conductor 1004 and the second input conductor 1006 comprise a bipolar output of an electrosurgical generator 1008. In the illustrated embodiment, a first intermediate conductor 1010 is electrically connected between the resistor R1 and the resistor R2; a second intermediate conductor 1012 is electrically connected between the resistor R2 and the resistor R3; a third intermediate conductor 1014 is electrically connected between the resistor R3 and the resistor R4; and a fourth intermediate conductor 1016 is electrically connected between the resistor R4 and the resistor R5.

In the illustrated embodiment, the first input conductor 1004 is electrically connected to a first electrode 1018, the second input conductor 1006 is electrically connected to a second electrode 1020, the first intermediate conductor 1010 is electrically connected to a first intermediate electrode 1022, the second intermediate conductor 1012 is electrically connected to a second intermediate electrode 1024, the third intermediate conductor 1014 is electrically connected to a third intermediate electrode 1026, and/or the fourth intermediate conductor 1016 is electrically connected to a fourth intermediate electrode 1028.

In some example embodiments, the resistors R1, R2, R3, R4, and R5 may have substantially equal electrical resistances. For example, each of the resistors R1, R2, R3, R4, and R5 may include a 100 Ohm resistor. Accordingly, some such embodiments may have substantially equal potential (e.g., voltage) differences between adjacent electrodes. In alternative embodiments, one or more of the resistors R1, R2, R3, R4, and R5 may have an electrical resistance substantially differing from at least one other of the resistors R1, R2, R3, R4, and R5.

In the illustrated embodiment, the voltage divider 1002 electrically interposes the electrosurgical generator 1008 and an electrosurgical device 1030 comprising the electrodes 1018, 1020, 1022, 1024, 1026, 1028. In some example embodiments, the voltage divider 1002 may be provided in an interface component configured to be releasably electrically connected between the electrosurgical generator 1008 and the electrosurgical device 1030. In some alternative embodiments, the voltage divider 1002 may be provided as part of the electrosurgical generator 1008 so that the voltage divider is electrically connected within the electrosurgical generator 1008 and the electrosurgical device 1030 is configured to be releasably electrically connected to the voltage divider 1002. In some alternative example embodiments, the voltage divider 1002 may be provided as part of the electrosurgical device 1030 so that the voltage divider 1002 is electrically connected within the electrosurgical device 1030 and the voltage divider 1002 is configured to the releasably electrically connected to the electrosurgical generator 1008.

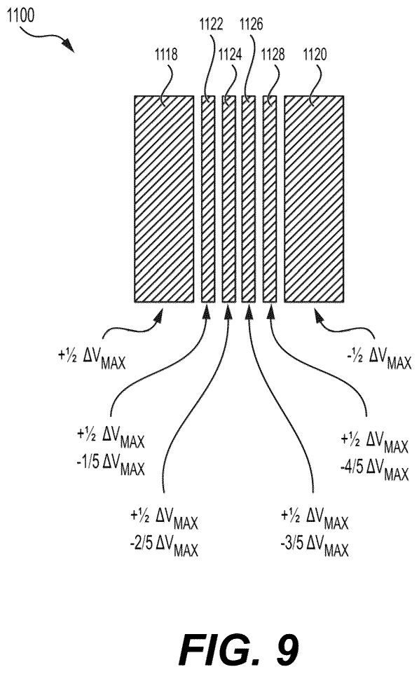

FIG. 9 is a simplified bottom view of an example electrode arrangement 1100 with annotations showing example maximum voltages, according to at least some aspects of the present disclosure. The electrode arrangement 1100 may be generally similar in structure and operation to other electrode arrangements and related components described herein, and repeated description of similar structures and operations is omitted for brevity. In the illustrated embodiment, the electrode arrangement 1100 includes a first electrode 1118, a second electrode 1120, a first intermediate electrode 1122, a second intermediate electrode 1124, a third intermediate electrode 1126, and/or a fourth intermediate electrode 1128, each of which is generally rectangular and which, together, form a generally rectangular array.

The respective potential (voltage) for each electrode 1118, 1120, 1122, 1124, 1126, 1128 is annotated in FIG. 9 for a maximum voltage difference between the first electrode 1118 and the second electrode 1120 of ΔV_max, which may correspond to a maximum voltage difference between a first input conductor 1004 and second input conductor 1006 supplied to the voltage divider 1002 of FIG. 8. The maximum voltage of the first electrode 1118 may be approximately +½ ΔV_max, the maximum voltage of the second electrode 1120 may be approximately −½ ΔV_max, the maximum voltage of the first intermediate electrode 1122 may be approximately +½ ΔV_max-⅕ ΔV_max, the maximum voltage of the second intermediate electrode 1124 may be approximately +½ ΔV_max-⅖ ΔV_max, the maximum voltage of the third intermediate electrode 1126 may be approximately +½ ΔV_max-⅗ ΔV_max, and/or the maximum voltage of the fourth intermediate electrode 1128 may be approximately +½ ΔV_max-⅘ ΔV_max. While these maximum voltages are based on resistors R1, R2, R3, R4, R5 having substantially equal electrical resistances, one of skill in the art will be able to calculate similar maximum voltages for alternative arrangements including resistors R1, R2, R3, R4, R5 having unequal electrical resistances.

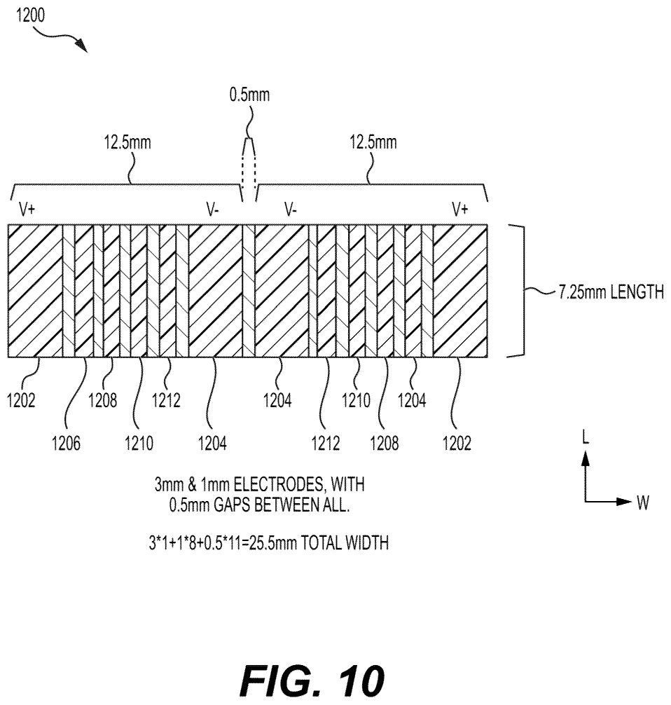

FIG. 10 is a simplified bottom view of an example electrode arrangement 1200 with annotations showing example dimensions, all according to at least some aspects of the present disclosure. The electrode arrangement 1200 may be generally similar in structure and operation to other electrode arrangements and related components described herein, and repeated description of similar structures and operations is omitted for brevity. In particular, the electrode arrangement 1200 is generally similar to that illustrated in FIGS. 1F and 1G and described above.

For clarity, various example embodiments may be described with reference to a length direction L and a width direction W. It will be understood that these designations are merely for consistency of description and are not intended to limit the scope of this disclosure to any particular orientation of an electrode arrangement with respect to other components of an electrosurgical device.

In the illustrated embodiment, the electrode arrangement 1200 includes a repeating configuration of a first electrode 1202, a second electrode 1204, and four intermediate electrodes 1206, 1208, 1210, 1212, each of which is generally rectangular and which, together, form a generally rectangular array. In this embodiment, the repeating configuration is arranged generally as a mirror-image, so that the second electrodes 1204 are nearest each other, thus avoiding adjacent electrodes having a V+/V− (e.g., ΔV_max) voltage differential. Although FIG. 10 illustrates an embodiment including two mirror-image configurations, it is within the scope of this disclosure to utilize any number of mirror-image configurations in a similar, generally repeating manner.

In the illustrated embodiment, the first electrodes 1202 and second electrodes 1204 are 3.0 mm in width and the intermediate electrodes 1206, 1208, 1210, 1212 are 1.0 mm in width. In the illustrated embodiment, the gaps between adjacent electrodes are 0.5 mm. Thus, the total width for the illustrated electrode arrangement is 25.5 mm. In the illustrated embodiment, the electrodes 1202, 1204, 1206, 1208, 1210, 1212 have equal lengths of 7.25 mm. It will be understood that these dimensions are merely exemplary and should not be considered limiting in any way.

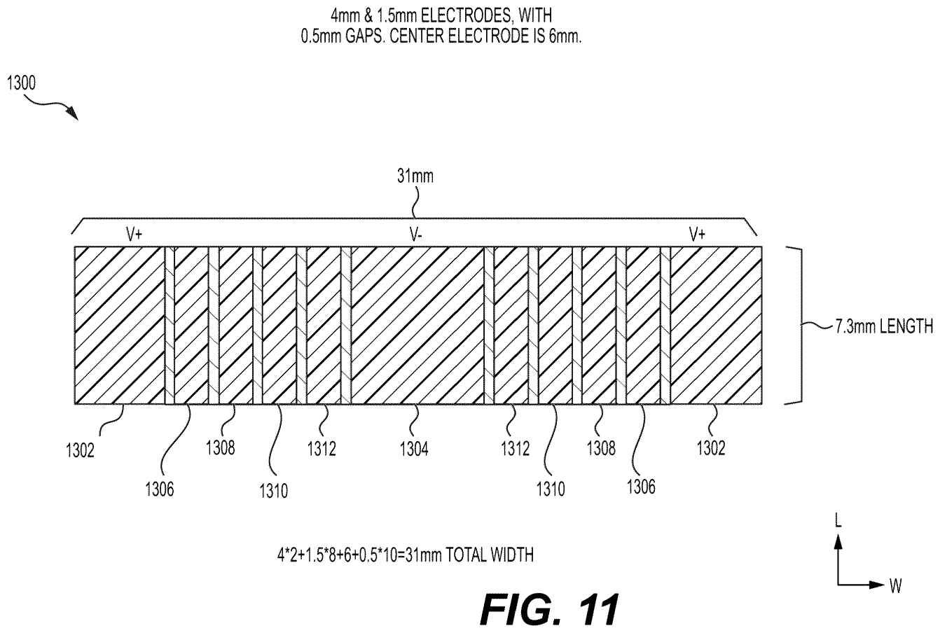

FIG. 11 is a simplified bottom view of an example electrode arrangement 1300 with annotations showing example dimensions, all according to at least some aspects of the present disclosure. The electrode arrangement 1300 may be generally similar in structure and operation to other electrode arrangements and related components described herein, and repeated description of similar structures and operations is omitted for brevity. In particular, the electrode arrangement 1300 is generally similar to that illustrated in FIG. 10 and described above.

In the illustrated embodiment, the electrode arrangement 1300 is similar to the mirror-image repeating configuration shown in FIG. 10, except that the adjacent second electrodes 1204 of FIG. 10 are replaced by a single, wider second electrode 1304 in the embodiment of FIG. 11 and avoid the gap between these electrodes 1204. Accordingly, the electrode arrangement 1300 of FIG. 11 includes a first electrode 1302 at each lateral end, a generally central, relatively wider second electrode 1304, and four intermediate electrodes 1306, 1308, 1310, 1312 in each repeated configuration, in a mirror-image arrangement. Although FIG. 11 illustrates an embodiment including two mirror-image configurations, it is within the scope of this disclosure to utilize any number of mirror-image configurations in a similar, generally repeating manner.

In the illustrated embodiment, the first electrodes 1302 are 4.0 mm in width, the second electrode 1304 is 6.0 mm in width, and the intermediate electrodes 1306, 1308, 1310, 1312 are 1.5 mm in width. In the illustrated embodiment, the gaps between adjacent electrodes are 0.5 mm. Thus, the total width for the illustrated electrode arrangement is 31.0 mm. In the illustrated embodiment, the electrodes 1302, 1304, 1306, 1308, 1310, 1312 have equal lengths of 7.3 mm. It will be understood that these dimensions are merely exemplary and should not be considered limiting in any way.

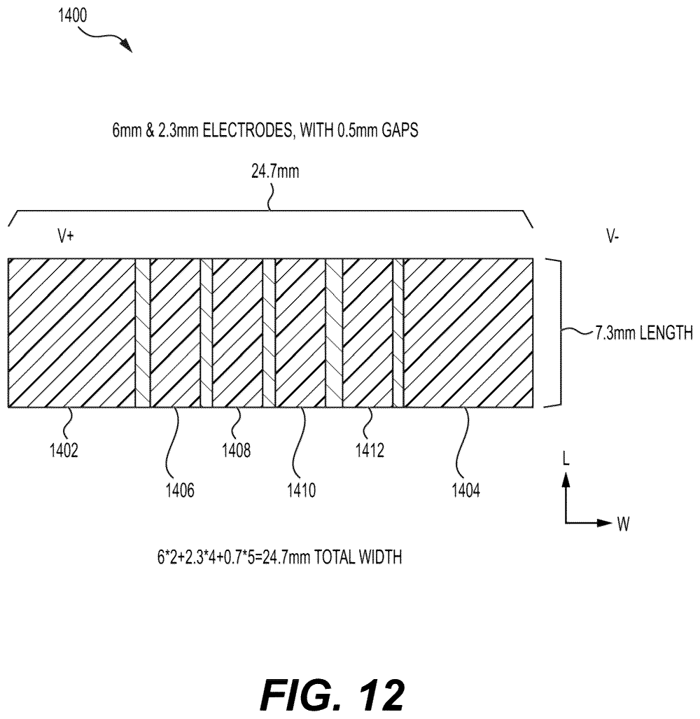

FIG. 12 is a simplified bottom view of an example electrode arrangement 1400 with annotations showing example dimensions, all according to at least some aspects of the present disclosure. The electrode arrangement 1400 may be generally similar in structure and operation to other electrode arrangements and related components described herein, and repeated description of similar structures and operations is omitted for brevity. In particular, the electrode arrangement 1400 is generally similar to that illustrated in FIGS. 1D and 1E and described above.

In the illustrated embodiment, the first electrode 1402 and second electrode 1404 are 6.0 mm in width and the intermediate electrodes 1406, 1408, 1410, 1412 are 2.3 mm in width. In the illustrated embodiment, the gaps between adjacent electrodes are 0.7 mm. Thus, the total width for the illustrated electrode arrangement is 24.7 mm. In the illustrated embodiment, the electrodes 1402, 1404, 1406, 1408, 1410, 1412 have equal lengths of 7.3 mm. It will be understood that these dimensions are merely exemplary and should not be considered limiting in any way.

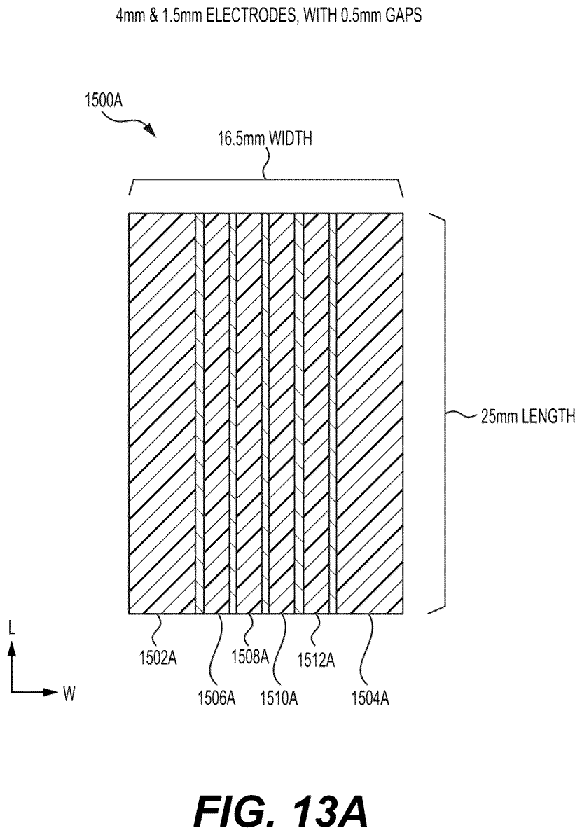

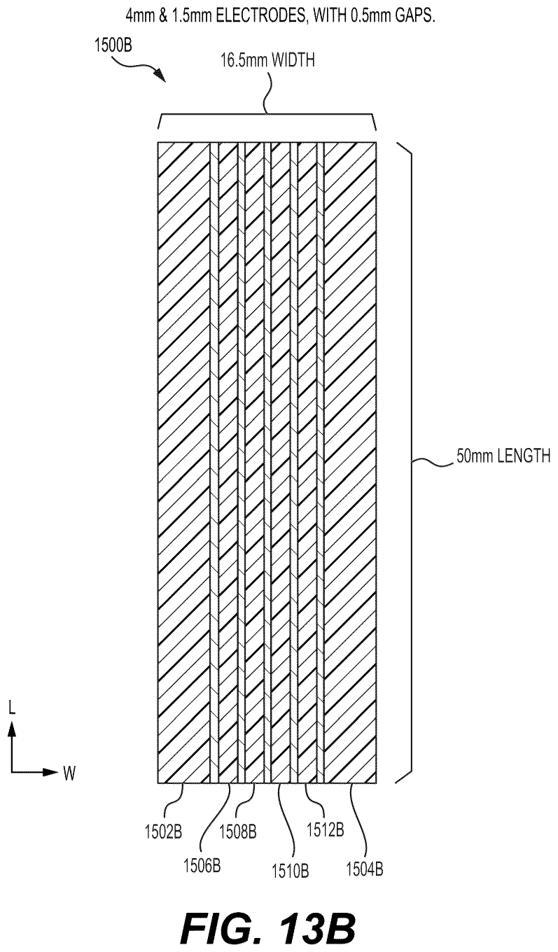

FIGS. 13A and 13B are a simplified bottom views of example electrode arrangements 1500A, 1500B with annotations showing example dimensions, all according to at least some aspects of the present disclosure. The electrode arrangements 1500A, 1500B may be generally similar in structure and operation to other electrode arrangements and related components described herein, and repeated description of similar structures and operations is omitted for brevity. In particular, the electrode arrangements 1500A, 1500B are generally similar to that illustrated in FIG. 12 and described above, except that the electrode arrangements 1500A, 1500B are substantially longer in length L. In particular, while each electrode of the embodiment of FIG. 12 is longer than it is wide, the overall electrode arrangement of FIG. 12 is wider than it is long. In contrast, in the embodiments of FIGS. 13A and 13B, each electrode is longer than it is wide and the overall electrode arrangements 1500A, 1500B are longer than they are wide.

In the illustrated embodiment, the first electrodes 1502A, 1502B and second electrodes 1504A, 1504B are 4.0 mm in width and the intermediate electrodes 1506A, 1506B, 1508A, 1508B, 1510A, 1510B, 1512A, 1512B are 1.5 mm in width. In the illustrated embodiment, the gaps between adjacent electrodes are 0.5 mm. Thus, the total width for the illustrated electrode arrangements is 16.5 mm. In the embodiment illustrated in FIG. 13A, the electrodes 1502A, 1504A, 1506A, 1508A, 1510A, 1512A have equal lengths of 25.0 mm. In the embodiment illustrated in FIG. 13B, the electrodes 1502B, 1504B, 1506B, 1508B, 1510B, 1512B have equal lengths of 50.0 mm. It will be understood that these dimensions are merely exemplary and should not be considered limiting in any way.

Generally, in some example embodiments according to at least some aspects of the present disclosure, the first and second electrodes may be about 4.0 mm wide, the intermediate electrodes may be about 0.5-1.5 mm wide, and the gaps between the electrodes may be about 0.5-1.5 mm, for a total width of about 15.0-20.0 mm. In similar alternative embodiments, these dimensions may vary by about +/−25% of these values.

Generally, in some example embodiments according to at least some aspects of the present disclosure, the first and second electrodes may be about 6.0 mm wide, the intermediate electrodes may be about 2.0-3.0 mm wide, and the gaps between the electrodes may be about 0.5-1.5 mm, for a total width of about 25.0-30.0 mm. In similar alternative embodiments, these dimensions may vary by about +/−25% of these values.

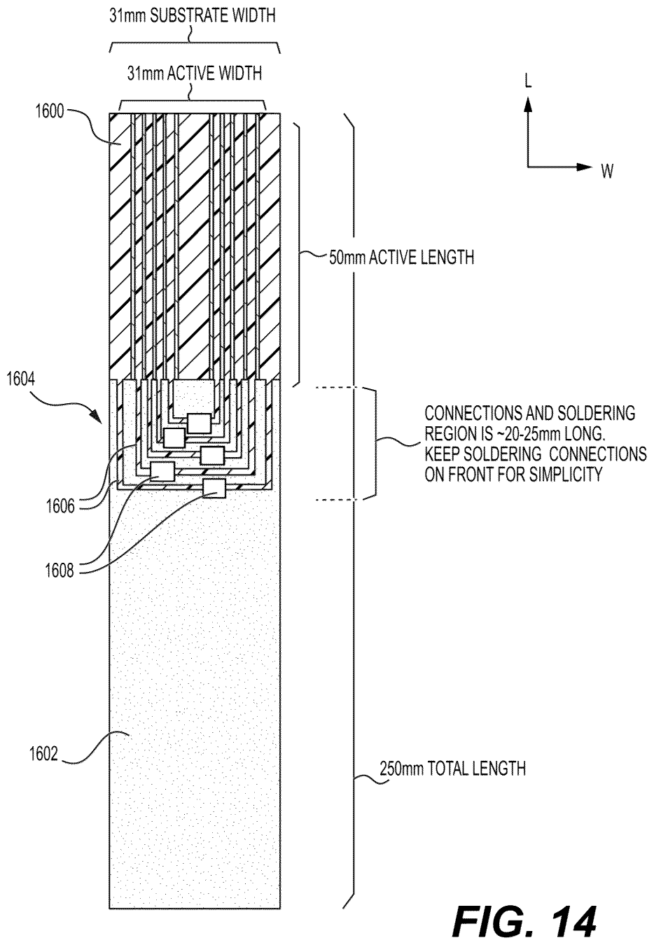

FIG. 14 is a simplified bottom view of an example electrode arrangement 1600 disposed on a substrate 1602, according to at least some aspects of the present disclosure. The electrode arrangement 1600 may be generally similar in structure and operation to other electrode arrangements and related components described herein, and repeated description of similar structures and operations is omitted for brevity. In particular, the electrode arrangement 1600 is generally similar to those illustrated in FIG. 11 (with the lengths L extended) and FIG. 13 (with the electrodes repeated) and described above.

In the illustrated embodiment, the substrate 1602 is 31.0 mm wide and 250.0 mm long, and the electrode arrangement 1600 is 31.0 mm wide and 50.0 mm long. In this embodiment, the electrode arrangement 1600 is disposed at one end of the substrate 1602.

Adjacent to the electrode arrangement 1600 on the substrate 1602 is a connections/soldering region 1604. The connections/soldering region 1604 includes a plurality of electrical conductors 1606 and soldering pads 1608. Generally, the electrical conductors 1606 are configured to electrically couple specific electrodes and soldering pads 1608 in a desired electrical configuration. The soldering pads 1608 are configured to facilitate solder connections to wires or other conductors, which may be electrically connected to an electrosurgical generator and/or sensing equipment, for example. In the illustrated embodiment, the connections/soldering region 1604 is approximately 20.0-25.0 mm long. In use, the electrode arrangement 1600 portion of the substrate 1602 is exposed, allowing contact with the target tissue. The remainder of the substrate 1602 (e.g., the portion including the connections/soldering region 1604) may be housed within an end effector or shaft, or may be otherwise protected and/or insulated from contact with the operative area.

In the illustrated embodiment, the electrode arrangement 1600 and the substrate 1602 are constructed in the form of a flexible printed circuit. The substrate may comprise Kapton® polyimide, for example, and/or the electrodes may be constructed from copper plated with nickel and gold, for example. In this example embodiment, the substrate may be about 0.05 mm thick and/or the electrodes may be about 0.036 mm thick. In some example embodiments, the substrate and/or electrodes may be generally flexible, such as to conform to other end effector components and/or to anatomical tissue.

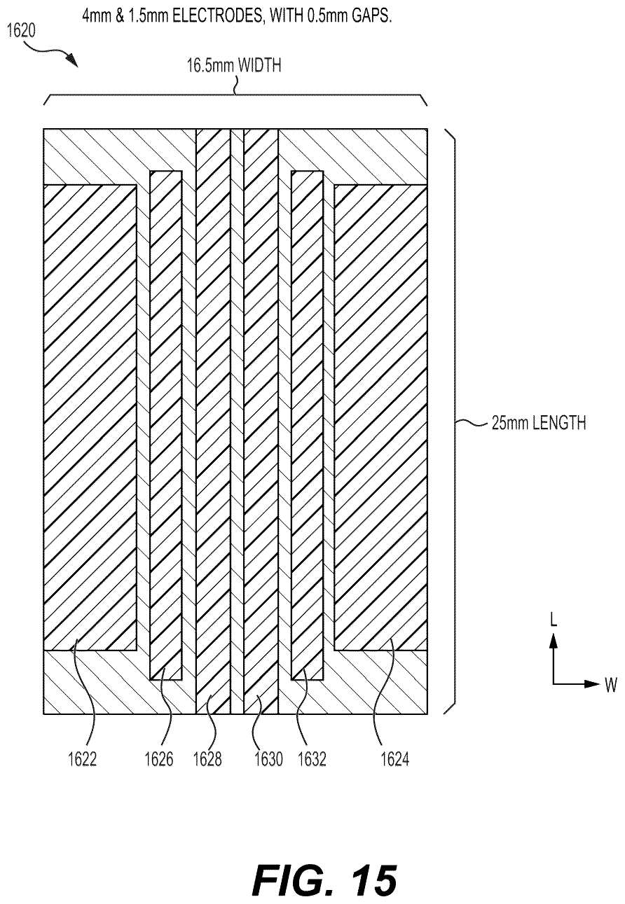

FIG. 15 is a simplified bottom view of an example electrode arrangement 1620 with annotations showing example dimensions, according to at least some aspects of the present disclosure. The electrode arrangement 1620 may be generally similar in structure and operation to other electrode arrangements and related components described herein, and repeated description of similar structures and operations is omitted for brevity. In particular, the electrode arrangement 1620 is generally similar to that illustrated in FIG. 13A and described above, except that the electrode arrangement 1620 includes at least one electrode having a different length, while all of the electrodes of the embodiment of FIG. 13A are substantially the same length.

In the illustrated embodiment, the first electrode 1622 and second electrode 1624 are 4.0 mm in width and the intermediate electrodes 1626, 1628, 1630, 1632 are 1.5 mm in width. In the illustrated embodiment, the gaps between adjacent electrodes are 0.5 mm. Thus, the total width for the illustrated electrode arrangement 1620 is 16.5 mm. In the illustrated embodiment, the first electrode 1622 and second electrode 1624 are shorter than the intermediate electrodes 1626, 1628, 1630, 1632. Additionally, in the illustrated embodiment, the intermediate electrodes 1626, 1632 nearest the first electrode 1622 and the second electrode 1624 are shorter than the centrally disposed intermediate electrodes 1628, 1630. That is, the centrally disposed intermediate electrodes 1628, 1630 are longest in length, and the outer, first and second electrodes 1622, 1624 are the shortest. In the illustrated embodiment, the centrally disposed intermediate electrodes 1628, 1630 are 25 mm long. Generally, the electrode dimensions may be selected to create a desired ablation shape. It will be understood that these dimensions are merely exemplary and should not be considered limiting in any way.

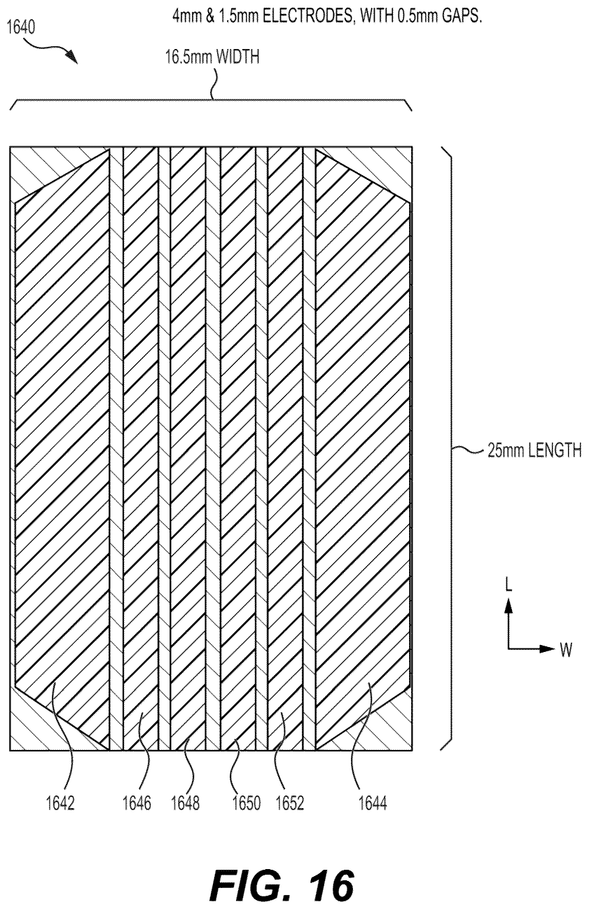

FIG. 16 is a simplified bottom view of an example electrode arrangement 1640 with annotations showing example dimensions, according to at least some aspects of the present disclosure. The electrode arrangement 1640 may be generally similar in structure and operation to other electrode arrangements and related components described herein, and repeated description of similar structures and operations is omitted for brevity. In particular, the electrode arrangement 1640 is generally similar to that illustrated in FIG. 13A and described above, except that the electrode arrangement 1640 includes at least one electrode having a different shape (e.g., generally trapezoidal), while all of the electrodes of the embodiment of FIG. 13A are generally rectangular.

In the illustrated embodiment, the first electrode 1642 and second electrode 1644 are 4.0 mm in width and the intermediate electrodes 1646, 1648, 1650, 1652 are 1.5 mm in width. In the illustrated embodiment, the gaps between adjacent electrodes are 0.5 mm. Thus, the total width for the illustrated electrode arrangement 1650 is 16.5 mm. In the illustrated embodiment, the first electrode 1652 and second electrode 1644 are generally trapezoidal, with the shorter of the parallel sides disposed outward away from the intermediate electrodes 1646, 1648, 1650, 1652. The intermediate electrodes 1646, 1648, 1650, 1652 are generally rectangular. In the illustrated embodiment, the electrodes are 25 mm long. Generally, the electrode shapes may be selected to create a desired ablation shape. It will be understood that these dimensions are merely exemplary and should not be considered limiting in any way.

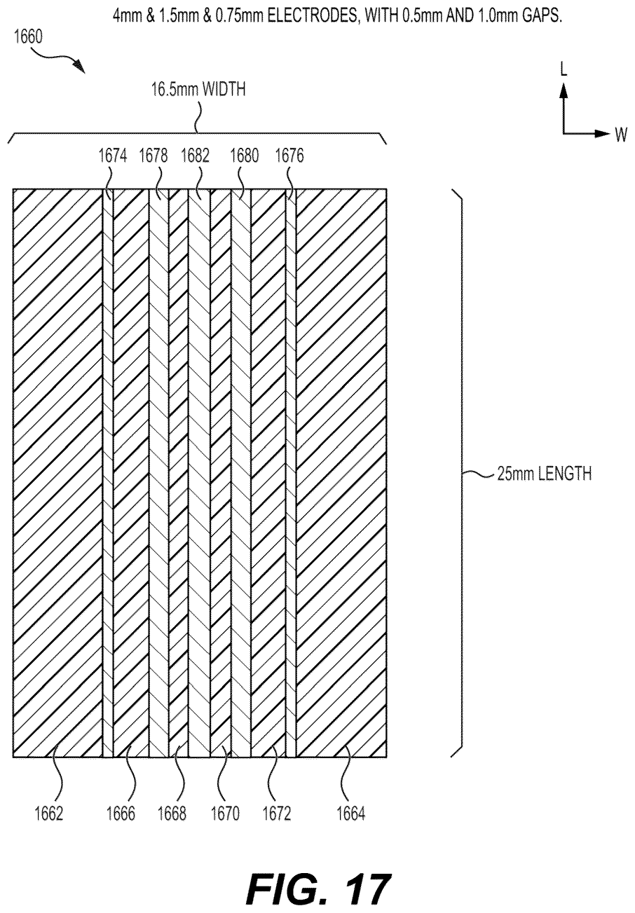

FIG. 17 is a simplified bottom view of an example electrode arrangement 1660 with annotations showing example dimensions, according to at least some aspects of the present disclosure. The electrode arrangement 1660 may be generally similar in structure and operation to other electrode arrangements and related components described herein, and repeated description of similar structures and operations is omitted for brevity. In particular, the electrode arrangement 1660 is generally similar to that illustrated in FIG. 13A and described above, except that the electrode arrangement 1660 includes at least one gap between electrodes having a different width, while all of the gaps of the embodiment of FIG. 13A are generally uniform, in addition to at least one intermediate electrode having a different width, while all of the intermediate electrodes had a uniform width of the embodiment of FIG. 13A.

In the illustrated embodiment, the first electrode 1662 and second electrode 1664 are 4.0 mm in width, the intermediate electrodes 1666, 1672 nearest the first electrode and second electrode are 1.5 mm in width, and the centrally disposed intermediate electrodes 1668, 1670 are 0.75 mm in width. In the illustrated embodiment, the gaps 1674, 1676 between the first electrode 1662 and second electrode 1624 and their respective adjacent intermediate electrodes 1666, 1672 are 0.5 mm. In the illustrated embodiment, the gaps 1678, 1680 between the outer intermediate electrodes 1666, 1672 and the centrally disposed intermediate electrodes 1668, 1670 are 1.0 mm. In the illustrated embodiment, the gap 1682 between the centrally disposed intermediate electrodes 1668, 1670 is 1.0 mm. Thus, the total width for the illustrated electrode arrangement 1660 is 16.5 mm. In the illustrated embodiment, the electrodes are 25 mm long. Generally, the gap widths may be selected to create a desired ablation shape. It will be understood that these dimensions are merely exemplary and should not be considered limiting in any way.

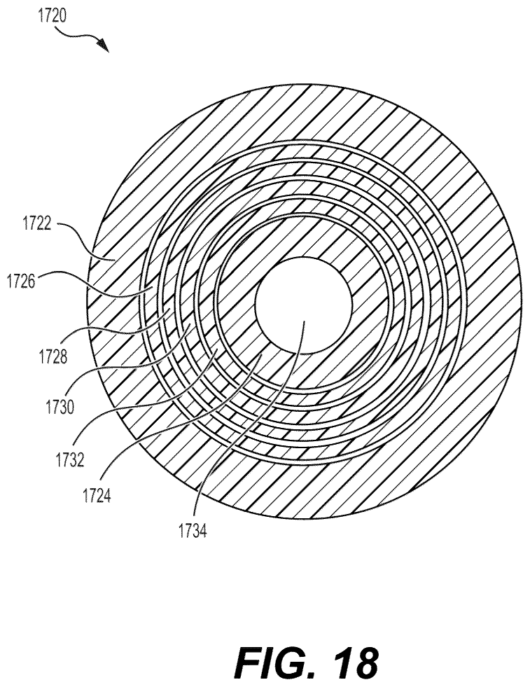

FIG. 18 is a simplified bottom view of an example nested, concentric electrode arrangement 1720, according to at least some aspects of the present disclosure. The electrode arrangement 1720 may be generally similar in structure and operation to other electrode arrangements and related components described herein, and repeated description of similar structures and operations is omitted for brevity. In particular, the electrode arrangement 1720 is generally similar to that illustrated in FIG. 5 and described above, except that the electrode arrangement 1720 includes a generally annular central electrode 1724, while the central electrode 500a of the embodiment of FIG. 5 is generally circular.

In the illustrated embodiment, a first (e.g., outer) electrode 1722 and a second (e.g., central) electrode 1724 are disposed concentrically. In the illustrated embodiment, four generally annular intermediate electrodes 1726, 1728, 1730, 1732 are disposed concentrically with and radially between the first electrode 1722 and second electrode 1724. In the illustrated embodiment, the electrode arrangement 1720 comprises a generally circular, central gap 1734, as well as annular gaps between adjacent pairs of electrodes.

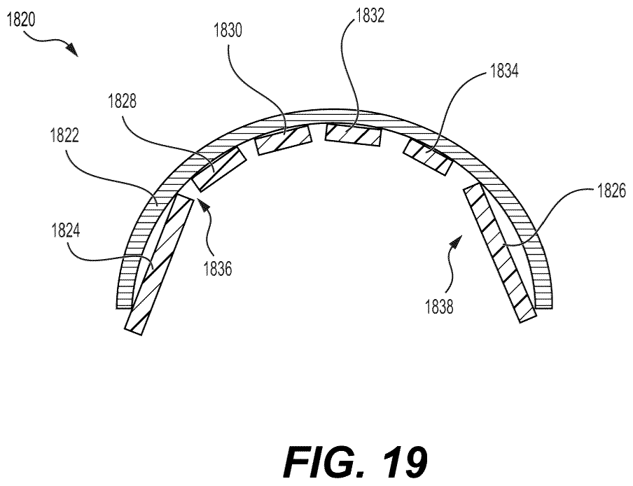

FIG. 19 illustrates a cross-section view of an example dome-shaped end effector 1820 with interior electrodes, according to at least some aspects of the present disclosure. In the illustrated embodiment, the end effector 1820 comprises a structural element 1822 and a plurality of electrodes 1824, 1826, 1828, 1830, 1832, 1834. The structural element 1822 forms a generally concave portion and the electrodes 1824, 1826, 1828, 1830, 1832, 1834 are disposed on an interior surface 1836 thereof. Thus, the electrodes provide a generally concave tissue-contacting surface 1838. The size and arrangement of the electrodes 1824, 1826, 1828, 1830, 1832, 1834 may be similar to other electrode arrangements described herein and repeated description is omitted for brevity. In some example embodiment, the structural element 1822 may be substantially rigid when subject to forces expected during an intended use. In some example embodiments, the structural element 1822 may be at least partially deformable (e.g., elastically and/or plastically) when subject to forces expected during an intended use.

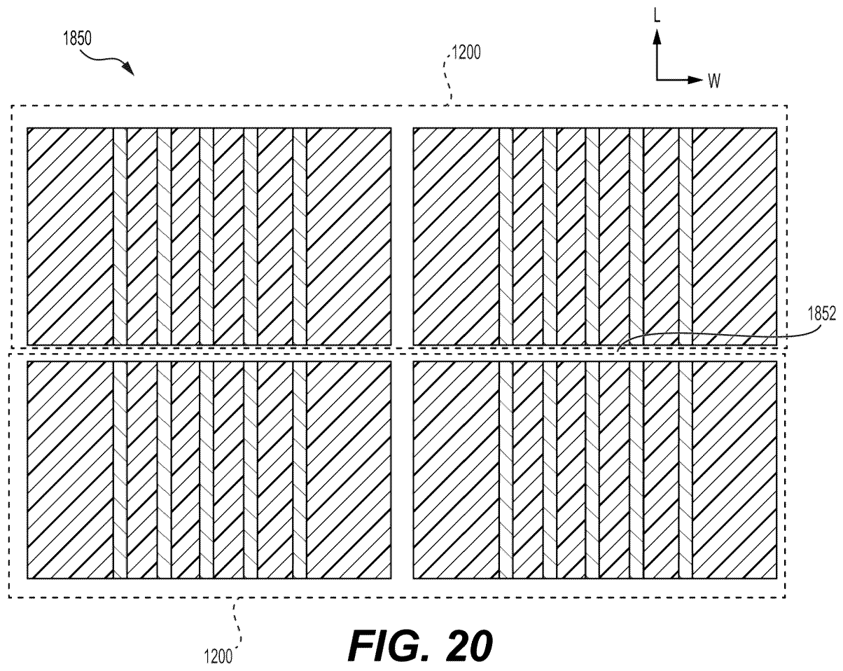

FIG. 20 illustrates a bottom view of an example tiled, rectangular electrode arrangement 1850, according to at least some aspects of the present disclosure. In the illustrated embodiment, the tiled arrangement comprises a first electrode arrangement 1200 (FIG. 10) disposed proximate a second electrode arrangement 1200 in the length direction. In the illustrated embodiment, a gap 1852 in the length direction interposes the first and second electrode arrangements 1200. Although FIG. 20 illustrates two electrode arrangements 1200 disposed in the length direction, alternative embodiments may include tiled arrangements of two or more other electrode arrangements widthwise offset and/or linearly offset and/or angularly offset, such as those disclosed herein.

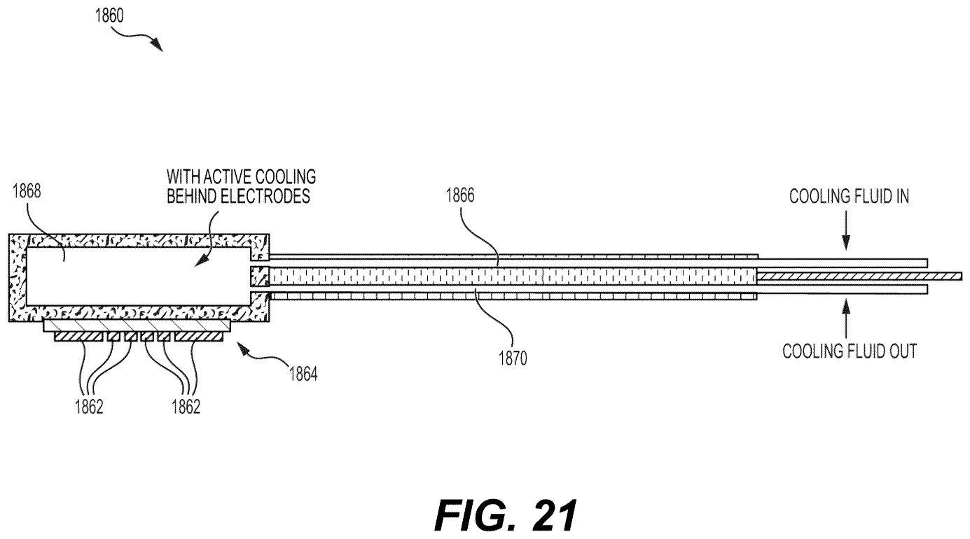

FIG. 21 illustrates a cross-section view of an example electrosurgical device 1860 configured for closed loop active cooling, according to at least some aspects of the present disclosure. In the illustrated embodiment, the electrosurgical device 1860 includes a plurality of electrodes 1862 disposed on an end effector 1864, generally as described elsewhere herein. A supply conduit 1866 is arranged to deliver cooling fluid to an interior chamber 1868 of the end effector 1864. A return conduit 1870 is arranged to direct cooling fluid away from the interior chamber 1868 of the end effector 1864. The end effector 1864 is constructed so that cooling fluid flowing through the interior chamber 1868 removes heat from the electrodes 1862. Some embodiments may include a thermal conductor, such as the thermal conductor 124 described above with reference to FIG. 1D, which may be arranged to conduct heat from the electrodes 1862 to the cooling fluid in the interior chamber 1868. Example cooling fluids may include, for example and without limitation, water and/or saline solution. The supply conduit 1866 and/or the return conduit 1870 may be operatively coupled to a source of cooling fluid and/or a receptacle for used cooling fluid. Alternatively, the return conduit 1870 may be omitted and cooling fluid may be expelled from the electrosurgical device 1860 and delivered to the surgical space.

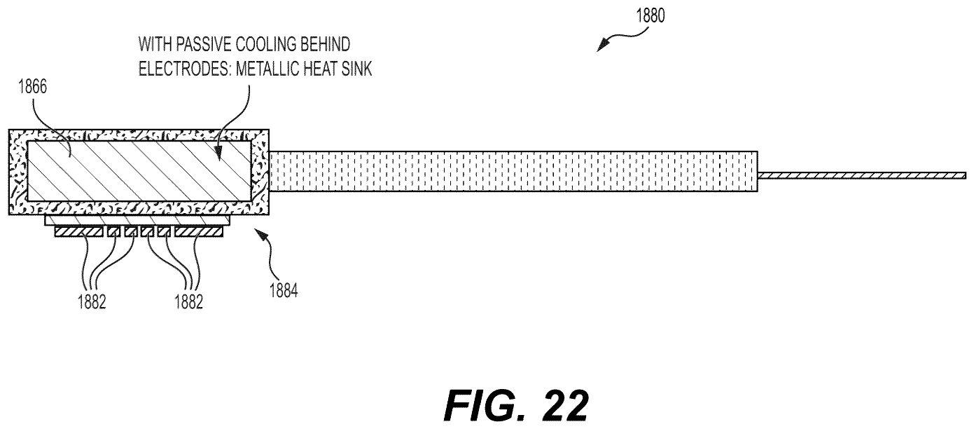

FIG. 22 illustrates a cross-section view of an example electrosurgical device 1880 configured for passive cooling, according to at least some aspects of the present disclosure. In the illustrated embodiment, the electrosurgical device 1880 includes a plurality of electrodes 1882 disposed on an end effector 1884, generally as described elsewhere herein. One or more heat sinks 1886 are disposed in thermal contact with the electrodes 1882 so that heat from the electrodes 1882 may flow into the heat sink 1886. In some example embodiments, the heat sink 1886 may be constructed from a solid material having a relatively high thermal mass and/or a relatively high thermal conductivity, such as a metal.

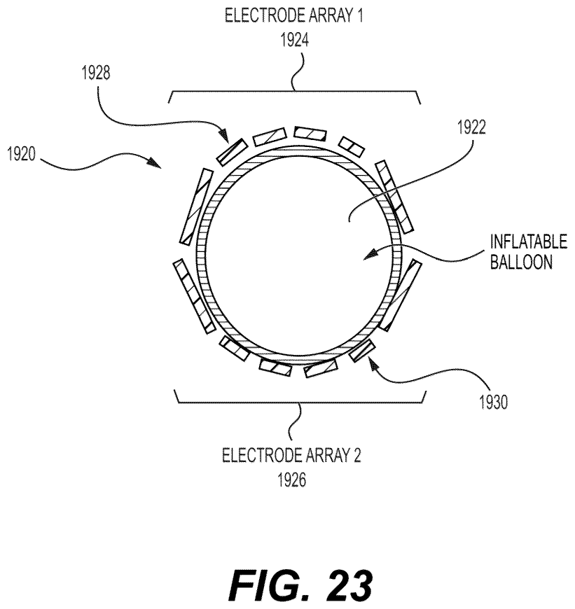

FIG. 23 illustrates a cross-section view of an example end effector 1920 including an expandable member in the form of an inflatable element 1922, according to at least some aspects of the present disclosure. In the illustrated embodiment, the end effector 1920 includes a first electrode array 1924 and a second electrode array 1926 disposed on one or more outer surfaces thereof. The end effector 1920 may be reconfigurable between a collapsed configuration and an expanded configuration (shown in FIG. 23), such as by inflation and/or deflation of the inflatable element 1922. The inflatable element 1922 may be disposed generally internally within the end effector 1920 and/or may act as a structural element of the end effector 1920. In the illustrated embodiment, each electrode array 1924, 1926 provides a respective generally convex tissue-contacting surface 1928, 1930 when the end effector is in the expanded configuration. The size and arrangement of the electrode arrays 1924, 1926 may be similar to other electrode arrangements described herein and repeated description is omitted for brevity.

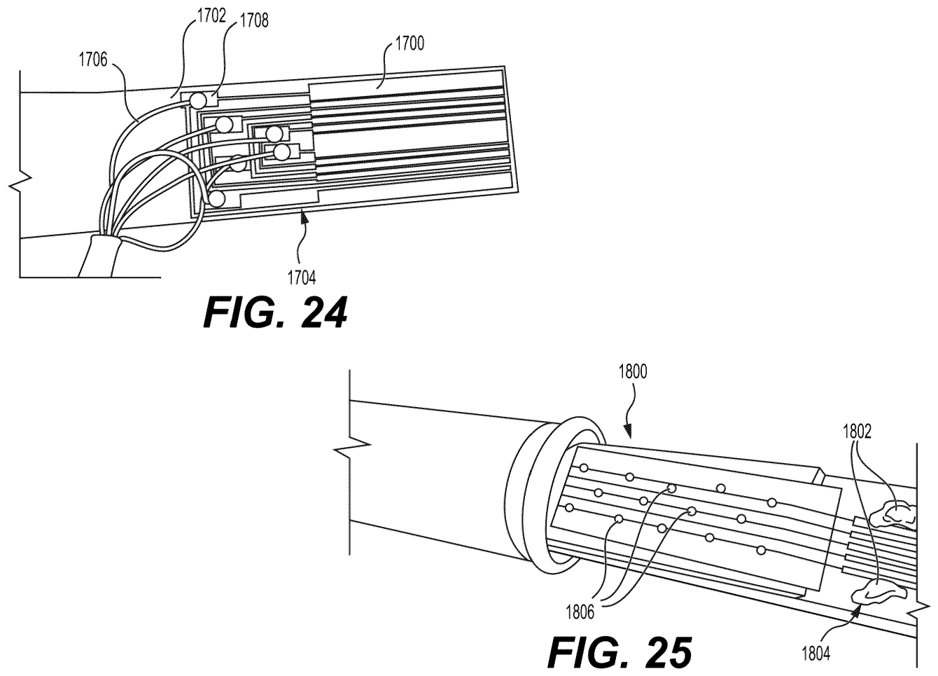

FIGS. 24-28 illustrate example embodiments including various optional features and configurations according to at least some aspects of the present disclosure. FIG. 24 illustrates a perspective view of an example generally rectangular electrode arrangement 1700 disposed on a substrate 1702, according to at least some aspects of the present disclosure. The electrode arrangement 1700 and substrate 1702 may be generally similar in structure and operation to other electrode arrangements and related components described herein, and repeated description of similar structures and operations is omitted for brevity. In particular, the electrode arrangement 1700 and substrate are generally similar to those illustrated in FIG. 14 and described above. In the illustrated embodiment, the connections/soldering region 1704 is shown with a respective wire 1706 soldered to each solder pad 1708.

FIG. 25 illustrates a perspective view of an example electrode arrangement 1800, according to at least some aspects of the present disclosure. The electrode arrangement 1800 may be generally similar in structure and operation to other electrode arrangements and related components described herein, and repeated description of similar structures and operations is omitted for brevity. In particular, the electrode arrangement 1800 substrate is generally similar to that illustrated in FIG. 13 and described above. Solder connections 1802 are visible in a connection/soldering region 1804 adjacent to the active electrode arrangement 1800. Additionally, the illustrated embodiment includes a plurality of suction openings 1806.

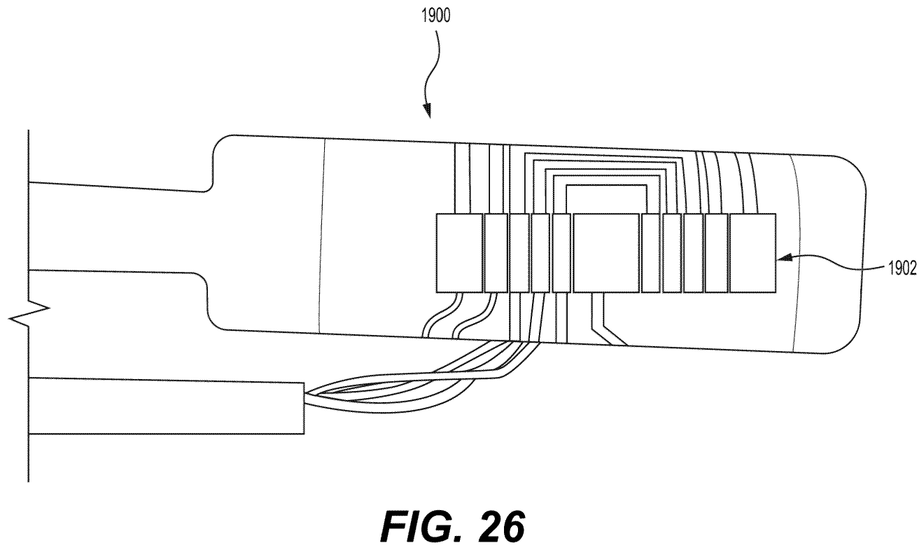

FIG. 26 illustrates a perspective view of an example end effector 1900 including an electrode arrangement 1902, according to at least some aspects of the present disclosure. The end effector 1900 may be generally similar in structure and operation to other end effectors and related components described herein, and repeated description of similar structures and operations is omitted for brevity. In particular, the electrode arrangement 1902 is generally similar to that illustrated in FIG. 11 and described above.

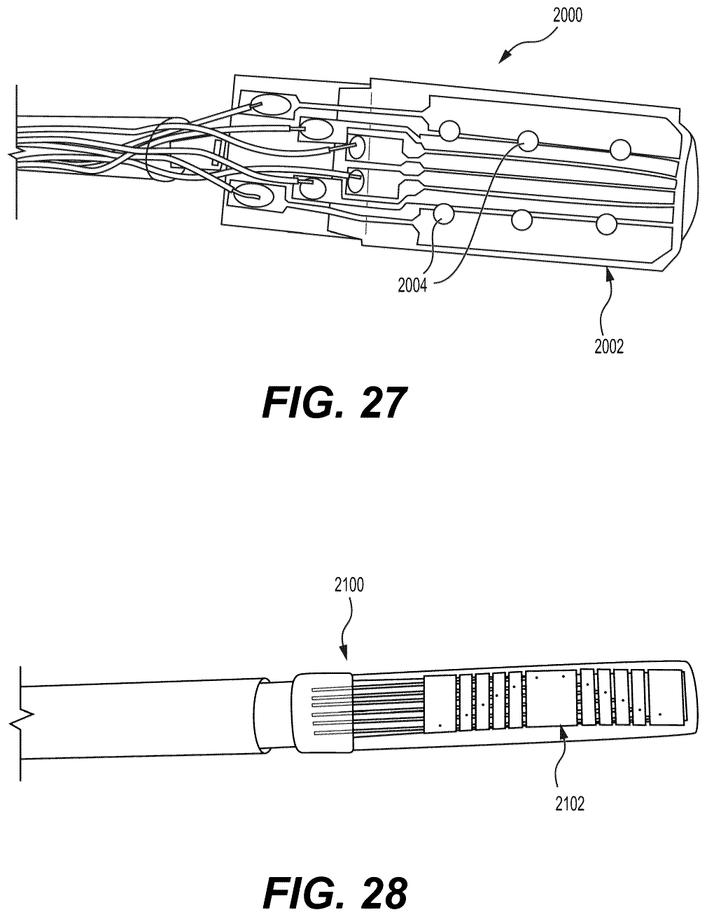

FIG. 27 illustrates a perspective view of an example end effector 2000 including an electrode arrangement 2002, according to at least some aspects of the present disclosure. The end effector 2000 may be generally similar in structure and operation to other end effectors and related components described herein, and repeated description of similar structures and operations is omitted for brevity. In particular, the electrode arrangement 2002 is generally similar to that illustrated in FIG. 13 and described above. Additionally, the illustrated embodiment includes a plurality of suction openings 2004. In the illustrated embodiment, the electrode arrangement 2002 may be about 17.0 mm by about 25.0 mm.

FIG. 28 illustrates a perspective view of an example end effector 2100 including an electrode arrangement 2102, according to at least some aspects of the present disclosure. The end effector 2100 may be generally similar in structure and operation to other end effectors and related components described herein, and repeated description of similar structures and operations is omitted for brevity. In particular, the electrode arrangement 2102 is generally similar to that illustrated in FIG. 11 and described above.

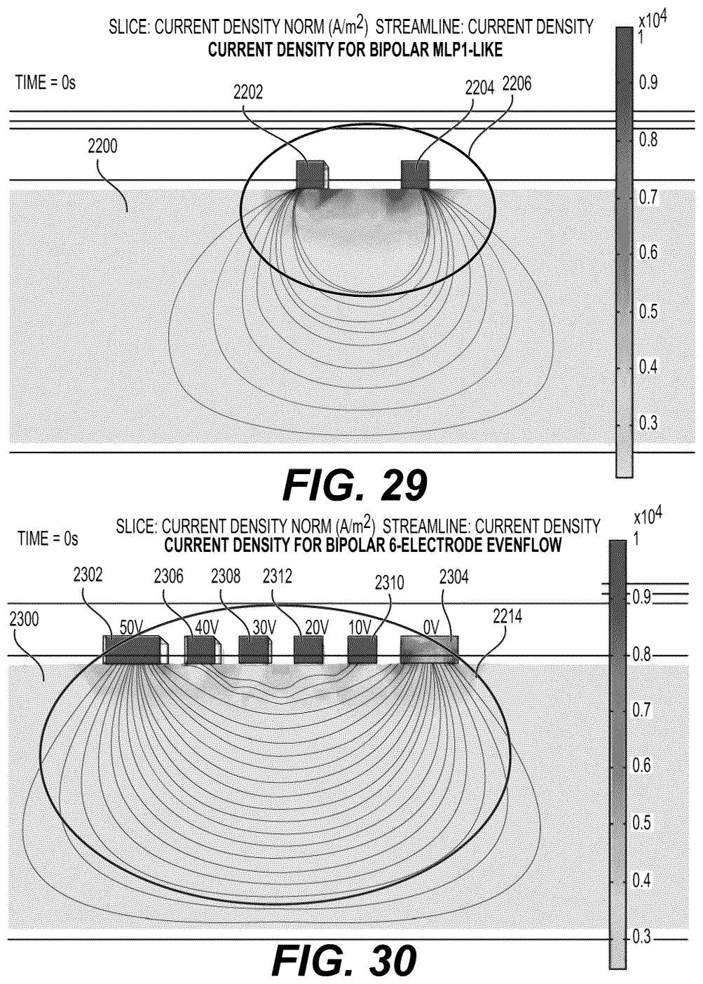

FIG. 29 illustrates a cross-sectional view of an example current density in a target tissue 2200 caused by a two-electrode 2202, 2204, bipolar ablation device. While the present disclosure contemplates that ablation devices with a configuration similar to that shown in FIG. 29 have been used safely and effectively, FIG. 29 shows that such devices may cause a high current density at the surface of the electrodes 2202, 2204 and near the tissue 2200 surface between the electrodes 2202, 2204. As a result, the effective ablation volume 2206 may be limited, such as to near the surface of the tissue 2200.

FIG. 30 illustrates a cross-sectional view of an example current density in a target tissue 2300 caused by an example ablation device including a first electrode 2302, a second electrode 2304, and four intermediate electrodes 2306, 2308, 2310, 2312, according to at least some aspects of the present disclosure. For example, the electrode arrangement illustrated in FIG. 30 may be generally similar to that illustrated in FIG. 12 and described above. As shown in FIG. 30, and as compared to the current density illustrated in FIG. 29, this example device causes a generally less concentrated current density near the tissue 2300 surface and enables a generally deeper penetration of current density. As a result, the effective ablation volume 2314 may be larger and/or may extend deeper into the tissue 2300. In general, for some embodiments according to at least some aspects of the present disclosure, a larger width between the electrodes 2302 and 2304 may lead to a deeper ablation, up to a point. If the electrodes are too far apart, then the current density may be too low to sufficiently heat the tissue. For example, to achieve approximately 5-15 mm tissue ablation depth, a width between the outermost points on the two outer electrodes (e.g., those with the largest voltage difference) may be between about 10 mm and about 30 mm when operating at between about 10 W and about 50 W power and about 400 and about 450 kHz AC, with some six electrode configurations similar to those shown in FIGS. 9-14.

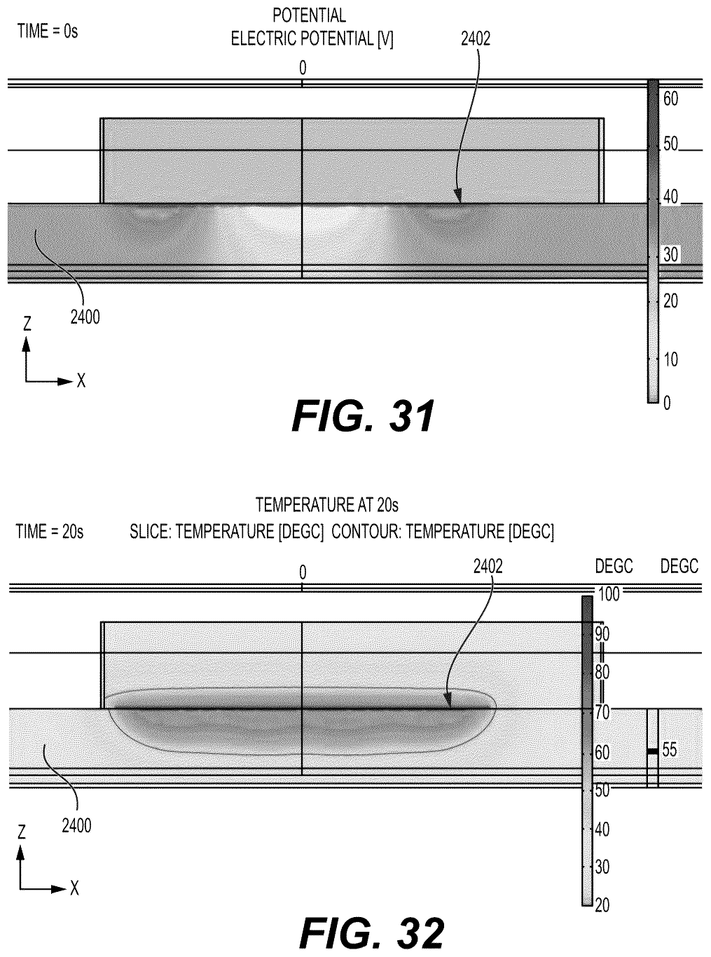

FIG. 31 illustrates a cross-sectional view of an example electrical potential in a target tissue 2400 caused by an example ablation device including an electrode arrangement 2402 generally similar to that shown in FIG. 10, and FIG. 32 illustrates a cross-sectional view of example temperature profile in the target tissue 2400 caused by an example ablation device including an electrode arrangement 2402 generally similar to that shown in FIG. 10, all according to at least some aspects of the present disclosure. Generally, as compared to an embodiment comprising a two-electrode, bipolar ablation device, the electrical potential and temperature variation are substantially more evenly distributed in the target tissue. That is, in a two-electrode, bipolar ablation device, the electrical potential and/or temperature variation may be more concentrated, such as at locations near the electrodes, as compared to an electrode arrangement 2402 generally similar to that shown in FIG. 10.

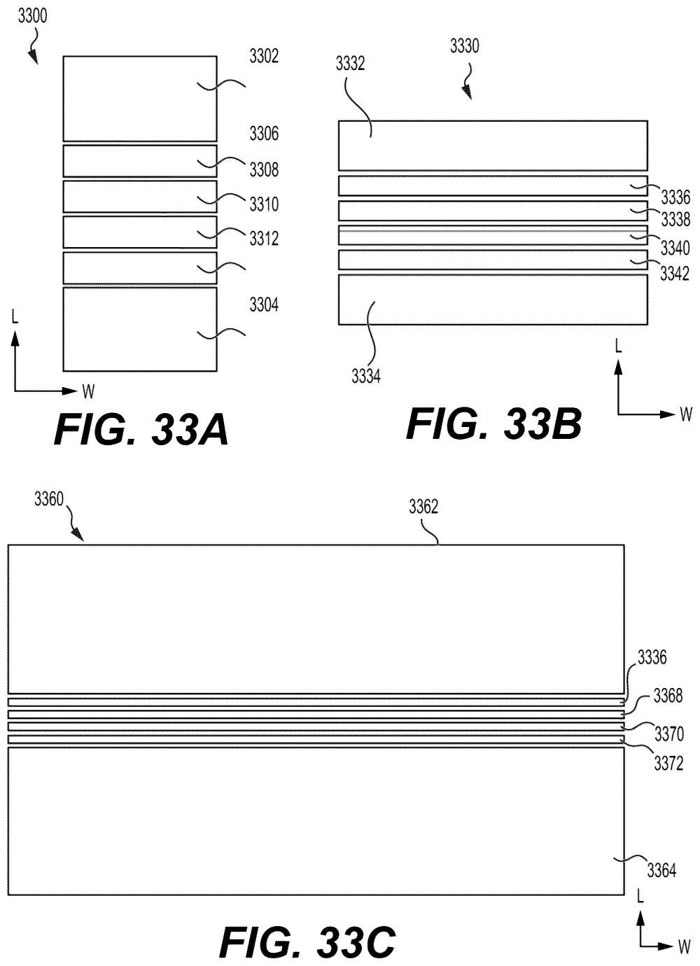

FIGS. 33A-33C illustrate alternative example electrode arrangements including generally rectangular electrodes disposed in generally rectangular arrays. These electrode arrangements may be generally similar in structure and operation to other electrode arrangements and related components described herein, and repeated description of similar structures and operations is omitted for brevity.

FIG. 33A is a simplified bottom view of an example electrode arrangement 3300, according to at least some aspects of the present disclosure. In the illustrated embodiment, the electrode arrangement 3300 comprises relatively wide (e.g., about 4 mm) first and second electrodes 3302, 3304 and four relatively narrow (e.g., about 1.5 mm) intermediate electrodes 3306, 3308, 3310, 3312 disposed therebetween. The spacing between the electrodes 3302, 3304, 3306, 3308, 3310, 3312 may be uniform at about 0.2 mm. The electrode arrangement 3300 may have a length of about 8 mm.

FIG. 33B is a simplified bottom view of an example electrode arrangement 3330, according to at least some aspects of the present disclosure. In the illustrated embodiment, the electrode arrangement 3330 comprises relatively wide (e.g., about 4 mm) first and second electrodes 3332, 3334 and four relatively narrow (e.g., about 1.5 mm) intermediate electrodes 3336, 3338, 3340, 3342 disposed therebetween. The spacing between the electrodes 3332, 3334, 3336, 3338, 3340, 3342 may be uniform at about 0.5 mm. The electrode arrangement 3330 may have a length of about 25 mm.

FIG. 33C is a simplified bottom view of an example electrode arrangement 3360, according to at least some aspects of the present disclosure. In the illustrated embodiment, the electrode arrangement 3360 comprises relatively wide (e.g., about 6 mm) first and second electrodes 3362, 3364 and four relatively narrow (e.g., about 0.5 mm) intermediate electrodes 3366, 3368, 3370, 3372 disposed therebetween. The spacing between the electrodes 3362, 3364, 3366, 3368, 3370, 3372 may be uniform at about 0.1 mm. The electrode arrangement 3360 may have a length of about 25 mm.

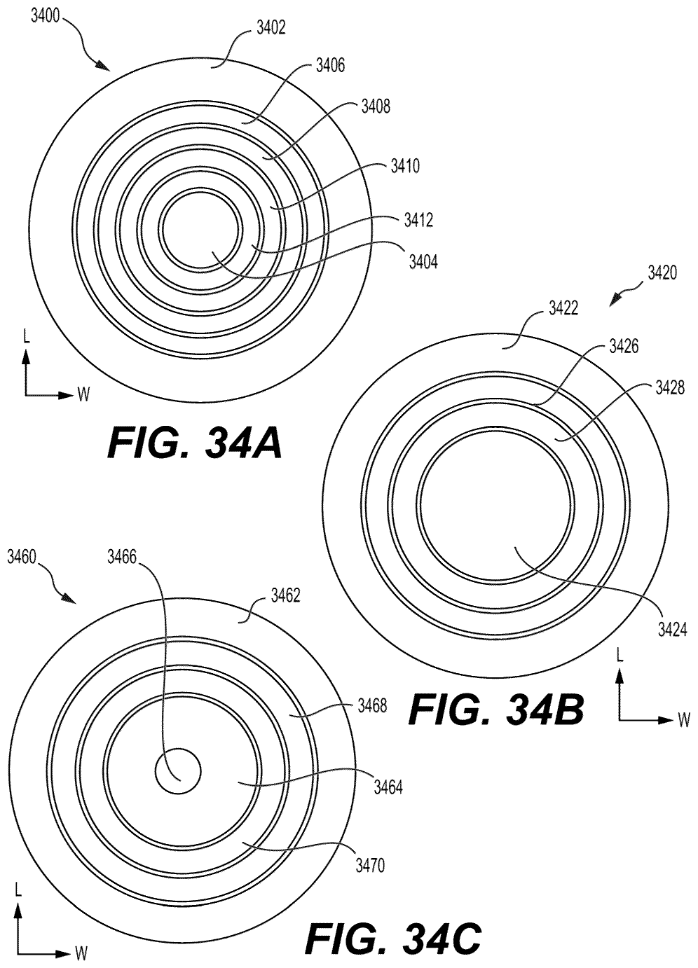

FIGS. 34A-34C illustrate alternative example electrode arrangements including nested, generally circular and/or annular ring electrodes. These electrode arrangements may be generally similar in structure and operation to other electrode arrangements and related components described herein, and repeated description of similar structures and operations is omitted for brevity.

FIG. 34A is a simplified bottom view of an example electrode arrangement 3400, according to at least some aspects of the present disclosure. In the illustrated embodiment, the electrode arrangement 3400 comprises a relatively wide (e.g., about 4 mm) outer, annular electrode 3402, a relatively wide (e.g., about 7 mm) circular inner electrode 3404, and four relatively narrow (e.g., about 1.5 mm) intermediate electrodes 3406, 3408, 3410, 3412 disposed therebetween. In this embodiment, the electrodes 3402, 3404, 3406, 3408, 3410, 3412 may be arranged concentrically, and the spacing between the electrodes 3402, 3404, 3406, 3408, 3410, 3412 may be uniform at about 0.2 mm. The electrode arrangement 3400 may have an overall diameter of about 29 mm.

FIG. 34B is a simplified bottom view of an example electrode arrangement 3420, according to at least some aspects of the present disclosure. In the illustrated embodiment, the electrode arrangement 3420 comprises a relatively wide (e.g., about 4 mm) outer, annular electrode 3422 separated by about 5-6 mm from a relatively wide (e.g., about 15.6 mm) circular inner electrode 3424, and two relatively narrow (e.g., about 2.5 mm) intermediate electrodes 3426, 3428 disposed therebetween. The ratio of the inner electrode 3424 surface area to the outer electrode 3422 surface area is about 1:2. In this embodiment, the electrodes 3422, 3424, 3426, 3428 may be arranged concentrically, and the spacing between the electrodes 3422, 3424, 3426, 3428 may be uniform at about 0.2 mm. The electrode arrangement 3420 may have an overall diameter of about 34.8 mm.

FIG. 34C is a simplified bottom view of an example electrode arrangement 3460, according to at least some aspects of the present disclosure. In the illustrated embodiment, the electrode arrangement 3460 comprises a relatively wide (e.g., about 4 mm) outer, annular electrode 3462, a relatively wide (e.g., about 15.6 mm) annular inner electrode 3464 with a central opening 3466 (e.g., about 4 mm), and two relatively narrow (e.g., about 2.5 mm) intermediate electrodes 3468, 3470 disposed therebetween. In this embodiment, the electrodes 3442, 3444, 3446, 3448 may be arranged concentrically, and the spacing between the electrodes 3442, 3444, 3446, 3448 may be uniform at about 0.2 mm. The electrode arrangement 3440 may have an overall diameter of about 34.8 mm.

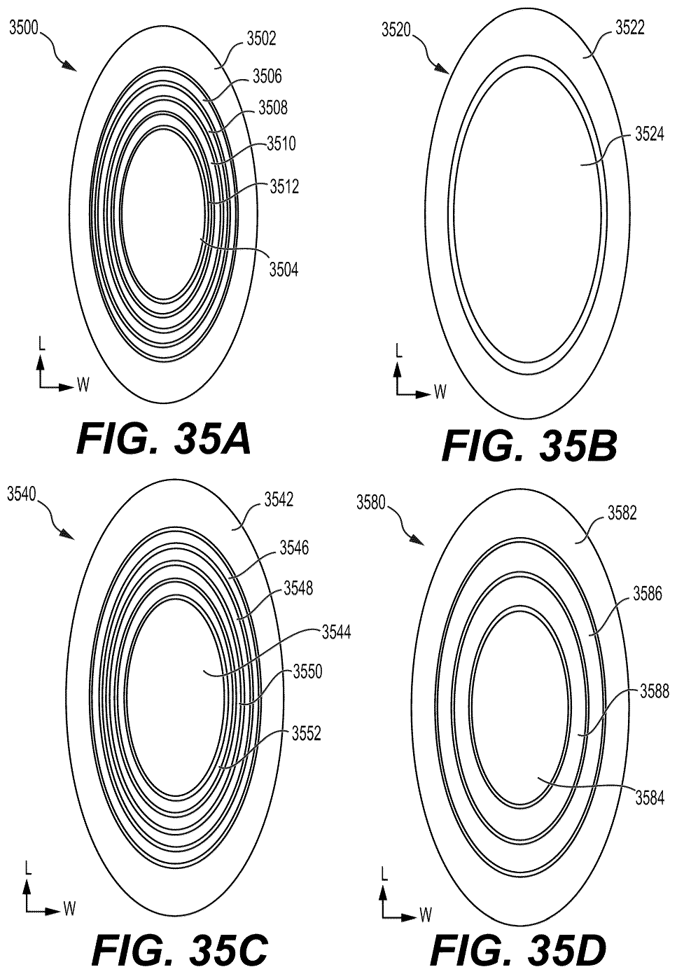

FIGS. 35A-35D illustrate alternative example electrode arrangements including nested, generally elliptical ring electrodes. These electrode arrangements may be generally similar in structure and operation to other electrode arrangements and related components described herein, and repeated description of similar structures and operations is omitted for brevity.

FIG. 35A is a simplified bottom view of an example electrode arrangement 3500, according to at least some aspects of the present disclosure. In the illustrated embodiment, the electrode arrangement 3500 comprises a relatively long (e.g., about 4 mm) outer elliptical electrode 3502, a relatively long (e.g., about 16.4 mm) elliptical inner electrode 3504, and four relatively narrow (e.g., about 1.5 mm) intermediate electrodes 3506, 3508, 3510, 3512 disposed therebetween. In this embodiment, the electrodes 3502, 3504, 3506, 3508, 3510, 3512 may be arranged concentrically, and the spacing between the electrodes 3502, 3504, 3506, 3508, 3510, 3512 may be uniform length at about 0.2 mm. In the width direction, the electrodes and electrode spacings are uniformly divided (scaled) by the same factor of 2. The electrode arrangement 3500 may have an overall length of about 38.4 mm and an overall width of about 19.2 mm.

FIG. 35B is a simplified bottom view of an example electrode arrangement 3520, according to at least some aspects of the present disclosure. In the illustrated embodiment, the electrode arrangement 3520 comprises a relatively long (e.g., about 4 mm) outer elliptical electrode 3522 and a relatively long (e.g., about 29.4 mm) elliptical inner electrode 3524, which may be arranged concentrically and spaced apart in the length direction by about 0.5 mm. In the width direction, the electrodes and electrode spacings are uniformly divided (scaled) by the same factor of 2. The electrode arrangement 3520 may have an overall length of about 38.4 mm and an overall width of about 19.2 mm.

FIG. 35C is a simplified bottom view of an example electrode arrangement 3540, according to at least some aspects of the present disclosure. In the illustrated embodiment, the electrode arrangement 3540 comprises the same dimensions as the electrode arrangement 35A in FIG. 35A, except all dimensions are multiplied by a factor of 1.25.

FIG. 35D is a simplified bottom view of an example electrode arrangement 3580, according to at least some aspects of the present disclosure. In the illustrated embodiment, the electrode arrangement 3580 comprises a relatively long (e.g., about 4 mm) outer elliptical electrode 3582, a relatively long (e.g., about 18 mm) elliptical inner electrode 3584, and two relatively narrow (e.g., about 3 mm) intermediate electrodes 3586, 3588 disposed therebetween. In this embodiment, the electrodes 3582, 3584, 3586, 3588 may be arranged concentrically, and the spacing between the electrodes 3582, 3584, 3586, 3588 may be uniform at about 0.3 mm. In the width direction, the electrodes and electrode spacings are uniformly divided (scaled) by the same factor of 2. The electrode arrangement 3580 may have an overall length of about 39.8 mm and an overall width of about 19.9 mm.

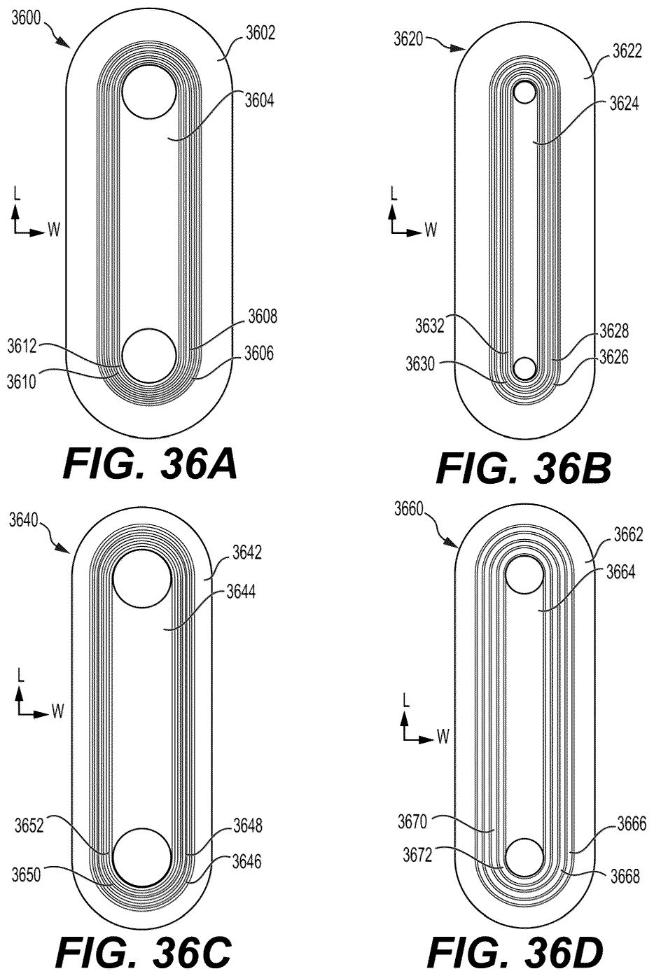

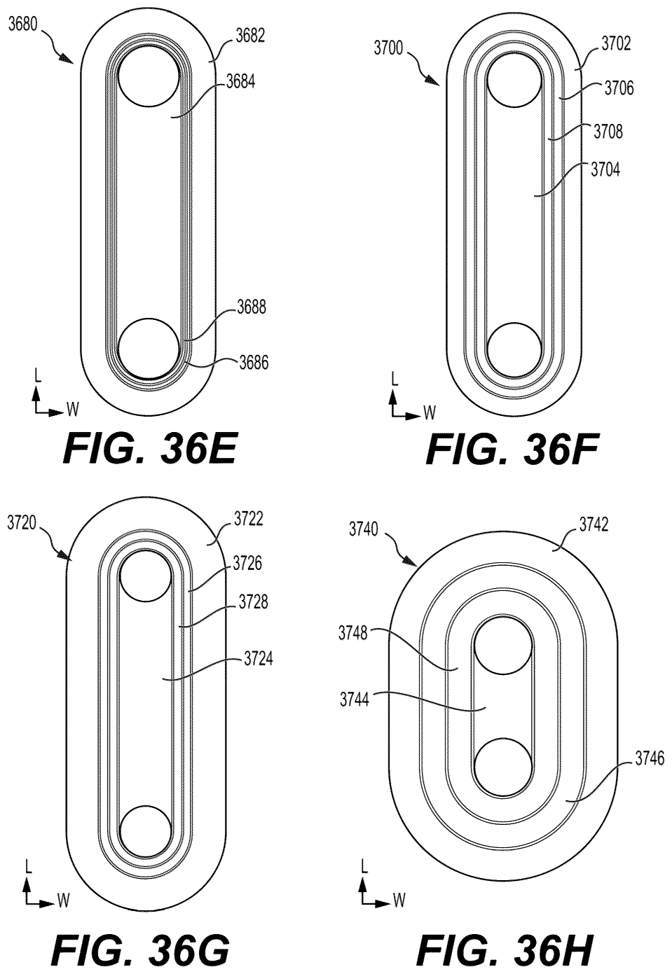

FIGS. 36A-36H illustrate alternative example electrode arrangements including nested, generally stadium-shaped ring electrodes. As used herein, “stadium-shaped” may describe a feature generally in the shape of a rectangle with semicircles at the opposite, short ends. These electrode arrangements may be generally similar in structure and operation to other electrode arrangements and related components described herein, and repeated description of similar structures and operations is omitted for brevity.

FIG. 36A is a simplified bottom view of an example electrode arrangement 3600, according to at least some aspects of the present disclosure. In the illustrated embodiment, the electrode arrangement 3600 comprises a relatively long (e.g., about 4 mm) outer stadium-shaped electrode 3602, a relatively long (e.g., about 19.4 mm) stadium-shaped inner electrode 3604 with a relatively narrow width (e.g., about 3 mm), and four relatively narrow (e.g., about 0.2 mm) intermediate electrodes 3606, 3608, 3610, 3612 disposed therebetween. In this embodiment, the electrodes 3602, 3604, 3606, 3608, 3610, 3612 may be arranged concentrically, and the spacing between the electrodes 3602, 3604, 3606, 3608, 3610, 3612 may be uniform at about 0.1 mm. The electrode arrangement 3600 may have an overall length of about 30 mm and an overall width of about 11.6 mm.

FIG. 36B is a simplified bottom view of an example electrode arrangement 3620, according to at least some aspects of the present disclosure. In the illustrated embodiment, the electrode arrangement 3620 comprises a relatively long (e.g., about 2 mm) outer stadium-shaped electrode 3622, a relatively long (e.g., about 17.4 mm) stadium-shaped inner electrode 3624 with a relatively narrow width (e.g., about 1.2 mm), and four relatively narrow (e.g., about 0.2 mm) intermediate electrodes 3626, 3628, 3630, 3632 disposed therebetween. In this embodiment, the electrodes 3622, 3624, 3626, 3628, 3630, 3632 may be arranged concentrically, and the spacing between the electrodes 3622, 3624, 3626, 3628, 3630, 3632 may be uniform at about 0.1 mm. The electrode arrangement 3620 may have an overall length of about 24 mm and an overall width of about 7.8 mm.

FIG. 36C is a simplified bottom view of an example electrode arrangement 3640, according to at least some aspects of the present disclosure. In the illustrated embodiment, the electrode arrangement 3640 comprises a relatively narrow (e.g., about 1 mm) outer stadium-shaped electrode 3642, a relatively long (e.g., about 19.4 mm) stadium-shaped inner electrode 3644 with a relatively large width (e.g., about 4 mm), and four relatively narrow (e.g., about 0.2 mm) intermediate electrodes 3646, 3648, 3650, 3652 disposed therebetween. In this embodiment, the electrodes 3642, 3644, 3646, 3648, 3650, 3652 may be arranged concentrically, and the spacing between the electrodes 3642, 3644, 3646, 3648, 3650, 3652 may be uniform at about 0.1 mm. The electrode arrangement 3640 may have an overall length of about 24 mm and an overall width of about 8.6 mm.

FIG. 36D is a simplified bottom view of an example electrode arrangement 3660, according to at least some aspects of the present disclosure. In the illustrated embodiment, the electrode arrangement 3660 comprises a relatively narrow (e.g., about 1.2 mm) outer stadium-shaped electrode 3662, a relatively long (e.g., about 18.2 mm) stadium-shaped inner electrode 3664 with a relatively narrow width (e.g., about 2 mm), and four relatively narrow (e.g., about 0.2 mm) intermediate electrodes 3666, 3668, 3670, 3672 disposed therebetween. In this embodiment, the electrodes 3662, 3664, 3666, 3668, 3670, 3672 may be arranged concentrically, and the spacing between the electrodes 3662, 3664, 3666, 3668, 3670, 3672 may be uniform at about 0.2 mm. The electrode arrangement 3660 may have an overall length of about 24.2 mm and an overall width of about 8 mm.

FIG. 36E is a simplified bottom view of an example electrode arrangement 3680, according to at least some aspects of the present disclosure. In the illustrated embodiment, the electrode arrangement 3680 comprises a relatively wide (e.g., about 1.5 mm) outer stadium-shaped electrode 3682, a relatively long (e.g., about 19.6 mm) stadium-shaped inner electrode 3684 with a relatively narrow width (e.g., about 4.5 mm), and two relatively narrow (e.g., about 0.1 mm) intermediate electrodes 3686, 3688 disposed therebetween. In this embodiment, the electrodes 3682, 3684, 3686, 3688 may be arranged concentrically, and the spacing between the electrodes 3682, 3684, 3686, 3688 may be uniform at about 0.2 mm. The electrode arrangement 3680 may have an overall length of about 24 mm and an overall width of about 8.9 mm.

FIG. 36F is a simplified bottom view of an example electrode arrangement 3700, according to at least some aspects of the present disclosure. In the illustrated embodiment, the electrode arrangement 3700 comprises a relatively wide (e.g., about 1 mm) outer stadium-shaped electrode 3702, a relatively long (e.g., about 19.2 mm) stadium-shaped inner electrode 3704 with a relatively narrow width (e.g., about 3 mm), and two relatively narrow (e.g., about 0.4 mm) intermediate electrodes 3706, 3708 disposed therebetween. In this embodiment, the electrodes 3702, 3704, 3706, 3708 may be arranged concentrically, and the spacing between the electrodes 3702, 3704, 3706, 3708 may be uniform at about 0.2 mm. The electrode arrangement 3700 may have an overall length of about 24 mm and an overall width of about 7.8 mm.

FIG. 36G is a simplified bottom view of an example electrode arrangement 3720, according to at least some aspects of the present disclosure. In the illustrated embodiment, the electrode arrangement 3720 comprises a relatively wide (e.g., about 2 mm) outer stadium-shaped electrode 3722, a relatively long (e.g., about 19.2 mm) stadium-shaped inner electrode 3724 with a relatively narrow width (e.g., about 3 mm), and two relatively narrow (e.g., about 0.4 mm) intermediate electrodes 3726, 3728 disposed therebetween. In this embodiment, the electrodes 3722, 3724, 3726, 3728 may be arranged concentrically, and the spacing between the electrodes 3722, 3724, 3726, 3728 may be uniform at about 0.2 mm. The electrode arrangement 3720 may have an overall length of about 26 mm and an overall width of about 9.8 mm.

FIG. 36H is a simplified bottom view of an example electrode arrangement 3740, according to at least some aspects of the present disclosure. In the illustrated embodiment, the electrode arrangement 3740 comprises a relatively wide (e.g., about 4 mm) outer stadium-shaped electrode 3742, a relatively long (e.g., about 23.6 mm) stadium-shaped inner electrode 3744 with a relatively large width (e.g., about 9 mm), and two relatively wide (e.g., about 3 mm) intermediate electrodes 3746, 3748 disposed therebetween. In this embodiment, the electrodes 3742, 3744, 3746, 3748 may be arranged concentrically, and the spacing between the electrodes 3742, 3744, 3746, 3748 may be uniform at about 0.2 mm. The electrode arrangement 3740 may have an overall length of about 44.8 mm and an overall width of about 21.2 mm.

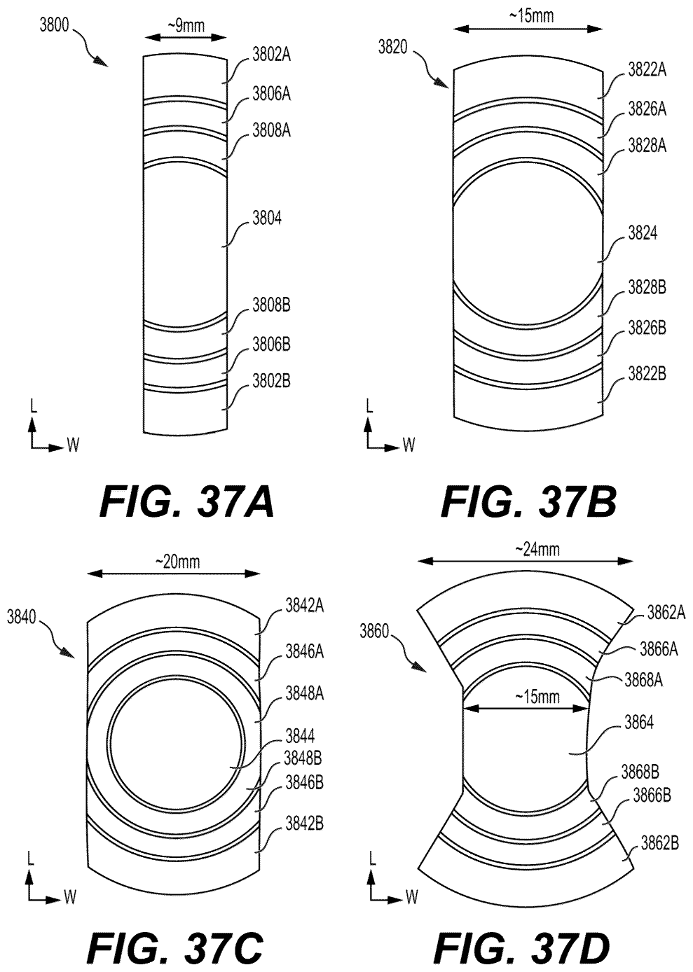

FIGS. 37A-37D illustrate alternative example electrode arrangements including truncated, nested, generally circular (or part circular) and/or annular (or semiannular) ring electrodes. These electrode arrangements may be generally similar in structure and operation to other electrode arrangements and related components described herein, and repeated description of similar structures and operations is omitted for brevity.

FIG. 37A is a simplified bottom view of an example electrode arrangement 3800, according to at least some aspects of the present disclosure. In the illustrated embodiment, the electrode arrangement 3800 comprises opposed, mirrored segments (e.g., semiannular segments) of a relatively wide (e.g., about 4 mm) outer, annular electrode 3802A, 3802B separated by about 5-6 mm from a segment of a relatively long (e.g., about 15.6 mm) circular inner electrode 3804, and opposed, mirrored segments (e.g., semiannular segments) of two relatively narrow (e.g., about 2.5 mm) annular intermediate electrodes 3806A, 3806B, 3808A, 3808B disposed therebetween. The ratio of the inner electrode 3804 surface area to the outer electrode 3802A, 3802B surface area is about 2:1. In this embodiment, the electrodes 3802A, 3802B, 3804, 3806A, 3806B, 3808A, 3808B may be arranged concentrically, and the spacing between the electrodes 3802A, 3802B, 3804, 3806A, 3806B, 3808A, 3808B may be uniform at about 0.2 mm. The electrode arrangement 3800 may have an overall length of about 34.8 mm and a width of about 9 mm.

FIG. 37B is a simplified bottom view of an example electrode arrangement 3820, according to at least some aspects of the present disclosure. In the illustrated embodiment, the electrode arrangement 3820 comprises opposed, mirrored segments of a relatively wide (e.g., about 4 mm) outer, annular electrode 3822A, 3822B separated by about 5-6 mm from a segment of a relatively long (e.g., about 15.6 mm) circular inner electrode 3824, and opposed, mirrored segments of two relatively narrow (e.g., about 2.5 mm) annular intermediate electrodes 3826A, 3826B, 3828A, 3828B disposed therebetween. The ratio of the inner electrode 3824 surface area to the outer electrode 3822A, 3822B surface area is about 1.4:1. In this embodiment, the electrodes 3822A, 3822B, 3824, 3826A, 3826B, 3828A, 3828B may be arranged concentrically, and the spacing between the electrodes 3822A, 3822B, 3824, 3826A, 3826B, 3828A, 3828B may be uniform at about 0.2 mm. The electrode arrangement 3820 may have an overall length of about 34.8 mm and a width of about 15 mm.

FIG. 37C is a simplified bottom view of an example electrode arrangement 3840, according to at least some aspects of the present disclosure. In the illustrated embodiment, the electrode arrangement 3840 comprises opposed, mirrored segments of a relatively wide (e.g., about 4 mm) outer, annular electrode 3842A, 3842B, a relatively long (e.g., about 15.6 mm) circular inner electrode 3844, and opposed, mirrored segments of two relatively narrow (e.g., about 2.5 mm) annular intermediate electrodes 3846A, 3846B, 3848A, 3848B disposed therebetween. In this embodiment, the electrodes 3842A, 3842B, 3844, 3846A, 3846B, 3848A, 3848B may be arranged concentrically, and the spacing between the electrodes 3842A, 3842B, 3844, 3846A, 3846B, 3848A, 3848B may be uniform at about 0.2 mm. The electrode arrangement 3840 may have an overall length of about 34.8 mm and a width of about 20 mm.

FIG. 37D is a simplified bottom view of an example electrode arrangement 3860, according to at least some aspects of the present disclosure. Unlike the embodiments of FIGS. 37A, 37B, and 37C, which are truncated in a generally linear, parallel manner, the embodiment of FIG. 37D is truncated into a bowtie shape. That is, the end portions have widths greater than a central portion. In the illustrated embodiment, the electrode arrangement 3860 comprises opposed, mirrored segments of a relatively wide (e.g., about 4 mm) outer, annular electrode 3862A, 3862B separated by about 5-6 mm from a segment of a relatively long (e.g., about 15.6 mm) circular inner electrode 3864, and opposed, mirrored segments of two relatively narrow (e.g., about 2.5 mm) annular intermediate electrodes 3866A, 3866B, 3868A, 3868B disposed therebetween. The ratio of the inner electrode 3864 surface area to the outer electrode 3862A, 3862B surface area is about 1:1. In this embodiment, the electrodes 3862A, 3862B, 3864, 3866A, 3866B, 3868A, 3868B may be arranged concentrically, and the spacing between the electrodes 3862A, 3862B, 3864, 3866A, 3866B, 3868A, 3868B may be uniform at about 0.2 mm. The electrode arrangement 3860 may have an overall length of about 34.8 mm and an overall width of about 24 mm. The narrower, central portion may have a width of about 15 mm.

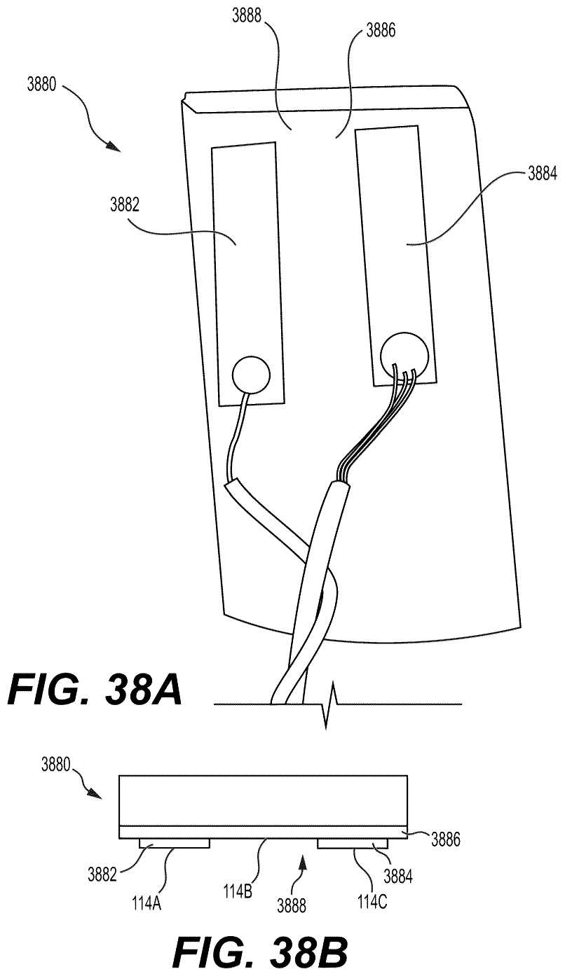

FIG. 38A is a bottom view of an example electrode arrangement 3880 including a semiconductor electrode interposing two outer electrodes, and FIG. 38B is a simplified elevation view of the embodiment of FIG. 38A; all according to at least some aspects of the present disclosure. In the illustrated embodiment, a first outer electrode 3882 and a second outer electrode 3884 are disposed in a spaced apart, generally parallel arrangement on and in electrical contact with a semiconductor substrate 3886. The tissue-contacting surface 3888 of the electrode arrangement 3880 therefore includes, from one side to the other, the first electrode 3882, the semiconductor substrate 3886, and the second electrode 3884. Although the illustrated embodiment includes two electrodes 3882, 3884, it will be understood that any electrode arrangement described herein may be constructed in a similar manner with semiconductor material(s) disposed in gap(s) between electrodes. In the illustrated embodiment, the first outer electrode 3882 includes the first tissue-contacting position 114A, the semiconductor substrate 3886 includes the intermediate tissue-contacting position 114B, and the second outer electrode 3884 includes the second tissue-contacting position 114C.

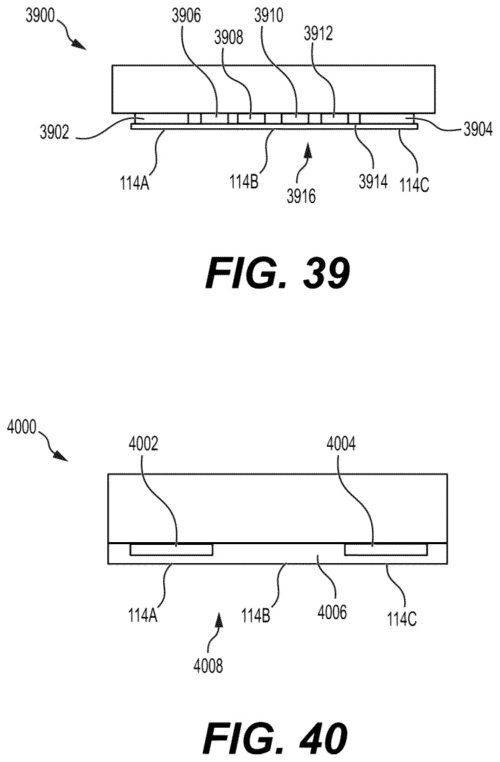

FIG. 39 is a simplified elevation view of an example electrode arrangement 3900 including a semiconductor electrode comprising a semiconductor layer disposed on metal conductors, according to at least some aspects of the present disclosure. In the illustrated embodiment, outer conductors 3902, 3904 and intermediate conductors 3906, 3908 3910, 3912 are disposed a manner generally similar to rectangular electrode arrays described elsewhere herein. In this embodiment, however, the conductor array is at least partially covered by a semiconductor layer 3914, which may present a tissue contacting surface 3916 and which may act as an electrode. For example, the semiconductor layer 3914 may be applied as a coating and/or a film, which may be electrically coupled to the conductors 3902, 3904, 3906, 3908 3910, 3912. In the illustrated embodiment, the semiconductor layer 3914 includes the first tissue-contacting position 114A, the intermediate tissue-contacting position 114B, and the second tissue-contacting position 114C.

FIG. 40 is a simplified elevation view of an example electrode arrangement 4000 including a semiconductor electrode with conductors embedded therein, according to at least some aspects of the present disclosure. In the illustrated embodiment, a first conductor 4002 and a second conductor 4004 are disposed in a spaced apart, generally parallel arrangement generally similar to the embodiment illustrated in FIGS. 38A and 38B. In the embodiment of FIG. 40, however, the conductors 4002, 4004 may be embedded within the semiconductor substrate 4006. The tissue-contacting surface 4008 of the electrode arrangement 4000 therefore includes the semiconductor substrate 4006, acting as an electrode, without the conductors 4002, 4004 being exposed on the tissue contacting surface 4008. In the illustrated embodiment, the semiconductor substrate 4006 includes the first tissue-contacting position 114A, the intermediate tissue-contacting position 114B, and the second tissue-contacting position 114C.

In some example embodiments including tissue contacting surfaces including semiconductor material(s), the semiconductor material(s) may be directly electrically connected to one or more conductors configured to deliver ablation energy. In some example embodiments, including tissue contacting surfaces including semiconductor material(s), the semiconductor material(s) may be electrically insulated from at least one conductor configured to deliver ablation energy. In some example embodiments, tissue contacting surfaces including semiconductor material(s), the semiconductor material(s) may include one or more gaps or breaks therein, which may at least partially electrically insulate at least one portion of the semiconductor material from another portion of the semiconductor material. In some example embodiments including tissue contacting surfaces including semiconductor material(s), the semiconductor material(s) may be uniformly shaped. In some example embodiments including tissue contacting surfaces including semiconductor material(s), the semiconductor material(s) may be non-uniformly shaped.

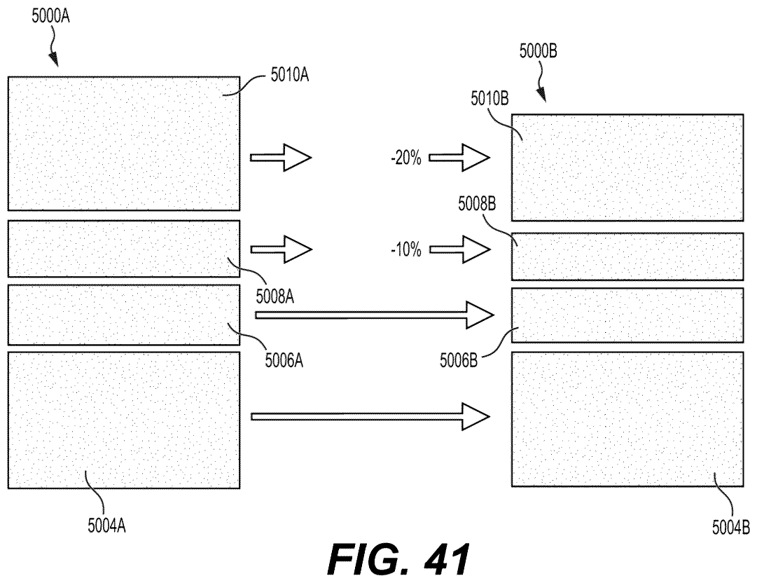

FIG. 41 is a simplified bottom view illustrating a comparison of symmetric 5000A and asymmetric 5000B electrode arrangements. In the illustrated embodiments, each electrode arrangement 5000A, 5000B includes relatively wide first outer electrodes 5004A, 5004B, relatively wide second outer electrodes 5010A, 5010B, and two relatively narrow intermediate electrodes 5006A, 5006B, 5008A, 5008B disposed therebetween. The symmetric arrangement 5000A and the asymmetric arrangement 5000B differ in that the width of the second electrode 5010B is reduced by about 20% as compared to the second electrode 5010A of the symmetric arrangement 5000A. Similarly, the width of the second intermediate electrode 5008B of the asymmetric arrangement 5000B is reduced by about 10% as compared to the second intermediate electrode 5008A of the symmetric arrangement 5004A. In this embodiment, the first intermediate electrodes 5006A, 5006B and the first outer electrodes 5004A, 5004B are substantially the same in the symmetric arrangement 5000A and the asymmetric arrangement 5000B. Generally, the asymmetric arrangement 5000B will produce an asymmetric ablation with hotter ablation proximate the narrower electrodes. Such an embodiment may be used, for example, to target specific anatomic features and/or to reduce heating at one end relative to the other. Similar asymmetry may be utilized in connection with any embodiment described herein, including rectangular arrays, concentric arrays, etc.

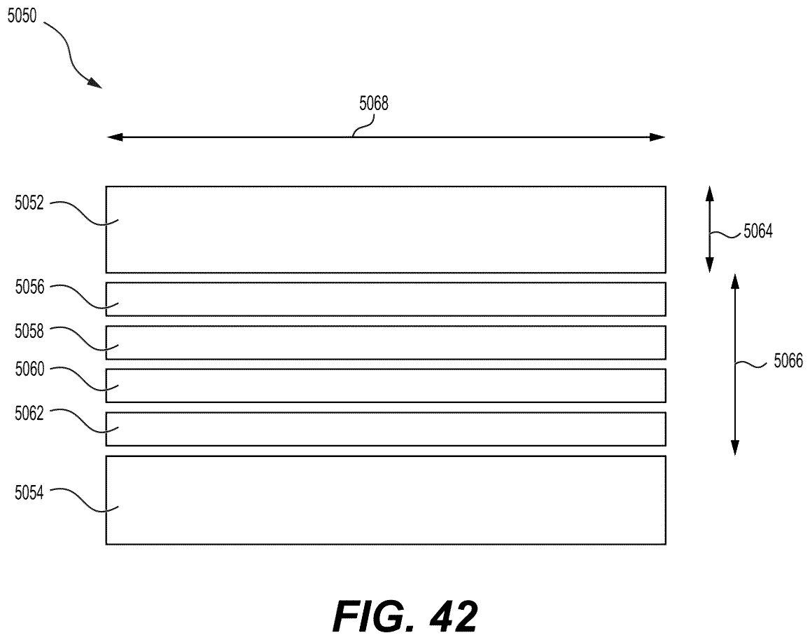

FIG. 42 is a simplified bottom view illustrating dimensions of a rectangular electrode array 5050 which may be varied. In the illustrated embodiment, the electrode arrangement 5050 includes relatively wide outer electrodes 5052, 5054 and four relatively narrow intermediate electrodes 5056, 5058, 5060, 5062 disposed therebetween. The outer electrodes 5052, 5054 have a width 5064 and are spaced apart by a separation 5066. The array 5050 has a length 5068.

In some example embodiments, an outer electrode width may be about 2-6 mm. In some configurations, an outer electrode width of greater than about 8 mm may cause excessively low current density at the electrodes, which may produce weak ablations. In some configurations, an outer electrode width of less than about 2 mm may cause excessively high current density at the electrodes, which may produce an excessively strong ablation.

In some example embodiments, such as those with an outer electrode width of about 2-6 mm, the separation 5066 may be about 2-8 mm. In some configurations, too narrow of a separation may cause excessive current density and/or overheating centrally between the electrodes. Too wide of a separation may cause bimodal current density and/or underheating centrally between the electrodes.

Generally, the presence of the intermediate electrodes 5056, 5058, 5060, 5062 reduces current density near the tissue surface, which reduces overheating near the tissue surface. Generally, the length 5068 may be arbitrarily changed; however, it may be advantageous to adjust the power density accordingly.

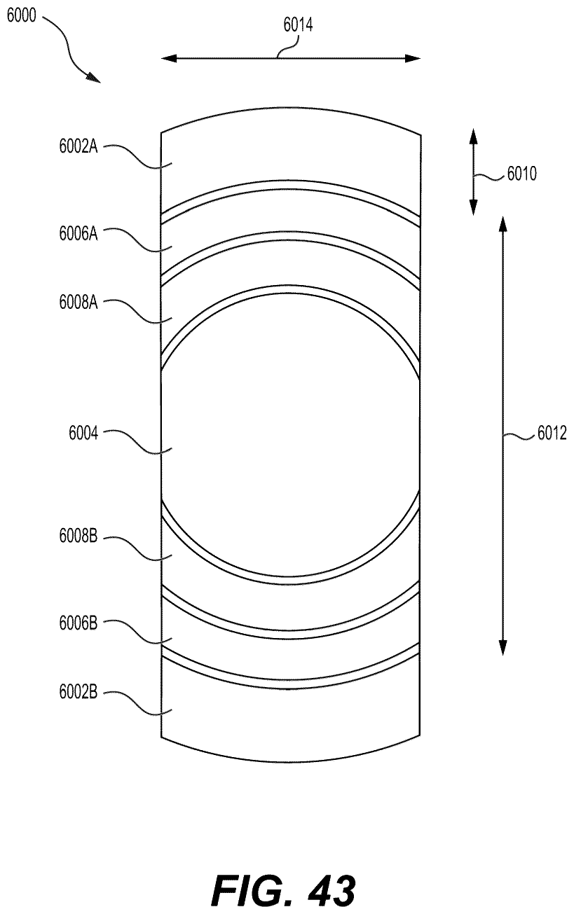

FIG. 43 is a simplified bottom view illustrating dimensions of a concentric electrode array 6000 which may be varied. In the illustrated embodiment, the electrode arrangement 6000 comprises opposed, mirrored segments of a relatively wide outer, annular electrode 6002A, 6002B, a segment of a relatively wide, circular inner electrode 6004, and opposed, mirrored segments of two relatively narrow annular intermediate electrodes 6006A, 6006B, 6008A, 6008B disposed therebetween. The outer electrodes 6002A, 6002B have a width 6010 and are spaced apart by a separation 6012. The array 6000 has a length 6014. The width 6010 and separation 6012 may generally be varied as described above with reference to the rectangular electrode arrangement 5050.

Generally, the presence of the intermediate electrodes 6006A, 6006B, 6008A, 6008B tends to increase the effective depth of the ablation, such as due to concentrated current density proximate the central region. Generally, the length 6014 may be arbitrarily changed; however, it may be advantageous to adjust the power density accordingly.

In some example embodiments, the power density of the applied ablation energy may be selected to produce consistent ablations. As used herein, “power density” may refer to the ratio of applied power over surface area of the outermost electrodes. For example, a power density of less than about 0.05 W/mm² may generally heat the target tissue slower than desired. A power density of about 0.05-0.5 W/mm² may generally produce desired ablation results. A power density greater than about 0.5 W/mm² may generally overheat the target tissue.

In some example embodiments, such as those with one inner electrode 6004 and two outer electrodes 6002A, 6002B, a ratio of the surface area of the inner electrode 6004 to the surface are of the outer electrodes 6002A, 6002B may be about 0.3:1 to about 3:1. In some configurations, a ratio of less than about 0.3:1 may tend to concentrate the ablation energy at the inner electrode. In some configurations, a ratio greater than about 3:1 may concentrate the ablation energy at the outer electrodes.

Referring back to FIGS. 1A-1E, an example method of creating a lesion 118 in a target tissue 116 may include one or more of the following operations, in any order. A tissue engagement portion 114 of an end effector 102 of an ablation device 100 may be positioned proximate a target tissue 116 so that a first tissue-contacting position 114A of the tissue engagement portion 114 is in electrical contact with the target tissue 116, an intermediate tissue-contacting position 114B of the tissue engagement portion 114 is in electrical contact with the target tissue 116, and a second tissue-contacting position 114C is in electrical contact with the target tissue 116. A lesion 118 may be created in the target tissue 116 by applying electrical ablation energy to the end effector 102 so that a magnitude of at least one electrical parameter differs between the first tissue-contacting position 114A, the intermediate tissue-contacting position 114B, and the second tissue-contacting position 114C so that an intermediate tissue-contacting position magnitude is between a first tissue-contacting position magnitude and a second tissue-contacting position magnitude.

In some embodiments, electrical ablation energy may be applied to a discrete first electrode 110a comprising the first tissue-contacting position 114A and a discrete second electrode 110f comprising the second tissue-contacting position 114C. In some embodiments, electrical ablation energy may be applied to a discrete intermediate electrode 110b, 110c, 110d, 110e comprising the intermediate tissue-contacting position 114B. In some embodiments, the intermediate electrode 110b, 110c, 110d, 110e may include at least two sequentially disposed, discrete intermediate electrodes 110b, 110c, 110d, 110e, and electrical ablation energy may be applied to the at least two sequentially disposed, discrete intermediate electrodes 110b, 110c, 110d, 110e so that the magnitude of the at least one electrical parameter differs incrementally between the at least two sequentially disposed, discrete intermediate electrodes 110b, 110c, 110d, 110e.

In some embodiments, such as in FIGS. 38A and 38B, electrical ablation energy may be applied to a semiconductor element 3886 comprising the intermediate tissue-contacting position.

In some embodiments, such as in FIG. 40, electrical ablation energy may be applied from an ablation energy source to a first electrical conductor 4002 and a second electrical conductor 4004, where the first electrical conductor 4002 is electrically coupled to the semiconductor element 4006 proximate the first tissue-contacting position 114A and the second electrical conductor 4004 is electrically coupled to the semiconductor element 4006 proximate the second tissue-contacting position 114C.

In some embodiments, such as in FIG. 39, electrical ablation energy may be applied to an intermediate electrical conductor 3906, 3908, 3910, 3912, which may be electrically coupled to the semiconductor element 3914 proximate the intermediate tissue-contacting position 114B. The magnitude of at least one electrical parameter may differ between the first electrical conductor 3902, the intermediate electrical conductor 3906, 3908, 3910, 3912, and the second electrical conductor 3904 so that the intermediate tissue-contacting position magnitude is between the first tissue-contacting position magnitude and the second tissue-contacting position magnitude.

In some embodiments, electrical ablation energy may be applied from the ablation source to the intermediate electrical conductor via a first resistor R1 and from the second electrical conductor via a second resistor R5.

In some embodiments, electrical ablation energy may be applied to a discrete first electrode 110a comprising the first tissue-contacting position 114A and a discrete second electrode 110f comprising the second tissue-contacting position 114C.

In some embodiments, the at least one electrical parameter may include electrical potential and/or electrical current. In some embodiments, electrical ablation energy may include radiofrequency electrical energy and/or pulsed field ablation electrical energy.

In some embodiments, the ablation device 100 may include a shaft 104 disposed proximally on the end effector 102, and the tissue engagement portion 114 of the end effector 102 of the ablation device 100 may be positioned using the shaft 104. In some embodiments, the ablation device 100 may include a handle 106 disposed proximally on the shaft 104, and the tissue engagement portion 114 of the end effector 102 of the ablation device 100 may be positioned using the handle 106. In some example embodiments, the ablation device may include at least one connecting element 108 configured to electrically couple the end effector 102 to an external ablation energy source, and electrical ablation energy may be applied to the end effector 102 via the at least one connecting element 108.

Some example embodiments configured for ablation of cardiac tissue according to at least some aspects of the present disclosure may be configured to ablate the target tissue more than about 5.0 mm in depth, such as 5.0-10.0 mm in depth.

An example embodiment including a first electrode, a second electrode, and four intermediate electrodes may be operated at about 30 to 80 V (maximum potential between the first electrode and the second electrode), about 300 to 500 kHz, and/or about 15 to 40 W.

An example procedure for creating a lesion that is between 3 and 15 mm deep would involve firmly touching the smaller electrode arrangement in FIG. 10, 11, or 12 to heart tissue, activating the source over a duration of 20 to 90 seconds at the conditions noted in the previous paragraph, and then turning off the source and removing the electrode. A second example procedure for creating a lesion that is between 3 and 15 mm deep would involve firmly touching the larger electrode arrangement in FIG. 13A, 13B, or 14 to heart tissue, activating the source over a duration of 40 to 300 seconds at the conditions noted in the previous paragraph, and then turning off the source and removing the electrode.

In various example embodiments according to at least some aspects of the present disclosure, devices may be configured to deliver energy to target tissue in a cautery format, a microwave format, a pulsed field ablation format, or a radiofrequency format, or any combination of any one or more of these. Example radiofrequency formats include bipolar, unipolar, and/or multipolar formats. In these cases, the voltage, current, power, and frequency may be different than what is noted elsewhere in this disclosure.

Some example embodiments according to at least some aspects of the present disclosure may be configured for use for operations other than and/or in addition to tissue ablation, such as testing operations. For example, and without limitation, some embodiments may be configured for cardiac pacing and/or sensing and/or for electroporation, such as over a relatively large area for drug delivery. Some non-ablation operations may be performed in connection with ablation operations, such as to assess the need for, the location of, and/or the efficacy of one or more ablations. Further, some ablation operations may be performed in connection with non-ablation operations, such as cut-and-ablate operations, clip-and-ablation operations, and/or cryogenic-treatment-and-ablation operations.

Although some example embodiments have been described above in the context of ablation of cardiac tissue, it will be understood that some alternative example embodiments may be utilized for use in connection with other target tissues and anatomical locations. For example, and without limitation, some alternative example embodiments may be configured for use in connection with target tissues associated with a patient's brain, gastrointestinal organs, lungs, liver, skin, gynecological organs, esophageal tissues, and/or tissues associated with the mouth and/or nose.

It is within the scope of this disclosure to perform electrosurgical (e.g., ablation) procedures using any suitable electrodes. For example, and without limitation, suitable electrode geometries may include rectangular configurations (e.g., generally parallel to the end effector and/or generally transverse to the end effector) and/or non-rectangular configurations (e.g., rings, concentric arrangements, bullseye configurations, generally circular arrangements, and/or generally elliptical arrangements), or any combination thereof (e.g., one or more lines inside an ellipse). In some example embodiments, one or more electrodes may have a three-dimensional configuration, such as a cup-shape, a dome-shape, a configuration generally conformable to the anatomy, and/or a custom-fit configuration for a particular anatomy. Other configurations may include a tiled matrix, a generally flat configuration in-plane, and/or a generally flat configuration out-of-plane. Some example electrodes may be in the form of discrete electrodes. Some example electrodes may be in the form of continuous electrodes, such as semiconductor electrodes, thin film conductors with various applied voltages, and/or conductive fluids.

Some example embodiments according to at least some aspects of the present disclosure may be configured to control multiple variables simultaneously in order to achieve desired performance during operation, where such coupled variables may include functions over time of electrode power, applied pressure for an inflatable device, applied vacuum for a suction device, and/or temperature as generated by ablations or from a secondary heating or chilling source, for example.

Some example embodiments according to at least some aspects of the present disclosure may be configured to address thermal considerations associated with operations. For example, some embodiments may be configured to remove excess heat actively and/or passively, and/or may be controlled to operate at a desired temperature. Some example embodiments may include cooling elements, such as heat pipes, which may be disposed proximate the electrodes, such as between the electrodes. Some example embodiments may utilize system-level cooling, such as cooling water pumped around and/or through electrodes and/or a tissue contacting surface. Some example embodiments may utilize electrode-level cooling, such as cooling water pumped behind electrodes. Some example embodiments may utilize passive cooling, such as one or more heat sinks disposed behind electrodes. Some example embodiments may include more than two electrodes configured for cooperative operation. For example, any number of electrodes may be configured for phased/switched groups.

It is within the scope of this disclosure to control electrical parameters associated with individual electrodes in any suitable manner. For example, and without limitation, various devices according to at least some aspects of the present disclosure may be configured to deliver electrical energy one or more electrodes with selected potential (voltage), current, and/or power. In some example embodiments, some electrical parameters may be configured for passive control, such as by using a resistor bank (e.g., resistive voltage divider), a capacitor bank, and/or an inductor bank. In some example embodiments, some electrical parameters may be configured for active control, such as individual electrode control, a switchbox configuration (e.g., one generator supplying multiple electrodes with active switching), multiple generators, and/or active monitoring and parameter adjustment. Some example embodiments may include control arrangements utilizing feedback, such as feedback pertaining to current, power, impedance, inductance, capacitance, temperature (e.g., tissue temperature), and/or time.

Although specific example embodiments have been described above, it is within the scope of this disclosure to configure devices in various alternative forms. For example, and without limitation, some example devices may include elements that are malleable, flexible (e.g., flexing in one plane, flexing in two planes, etc.), and/or rigid. Some example devices may include elements that are configured for rolling and/or folding, such as rolling on along a short axis and/or rolling along a long axis.

It is within the scope of this disclosure to utilize various methods of fixation in connection with example embodiments. For example, and without limitation, some example devices may be configured to utilize manual fixation (e.g., operator-applied mechanical load), vacuum fixation, clamping, and/or magnetic coupling to hold an end effector against a target tissue. See, for example, the description above referring to FIGS. 2 and 3.

It is within the scope of this disclosure to utilize end effectors having various shapes. For example, and without limitation, some example end effectors may be contoured and/or may include electrodes disposed generally on the inside and/or the outside. In some example embodiments, electrodes may be generally round, ring-shaped, provided as a jacket or collar, generally tubular or cylindrical (e.g., full radius and/or partial radius).

It is within the scope of this disclosure to utilize end effectors including inflatable elements. In some example embodiments, components including inflatable elements may be configured to conform to adjacent structures (e.g., anatomical structures). In some example embodiments, components including inflatable elements may be configured to have a predetermined, generally fixed shape. See, for example, the description above referring to FIGS. 4, 19, and 23.

It is within the scope of this disclosure to conduct procedures involving any suitable access approach. For example, endocardial access may be obtained using percutaneous approaches (e.g., arterial and/or venous) and/or surgical approaches (e.g., transapical, fem-fem bypass (venous), fem-fem bypass (arterial), bypass cannula traditional (arterial), bypass cannular traditional (venous), and/or atriotomy). Epicardial access may be obtained using percutaneous approaches (e.g., sub-xiphoid) and/or surgical approaches (e.g., lateral (right or left), surgical window, and/or sternotomy (full or partial)), and/or minimally invasive surgical approaches (MIS), for example. It will be understood that the foregoing list is merely exemplary and is not to be considered limiting.

It is within the scope of this disclosure to conduct procedures involving any portions of the heart using apparatus and/or methods disclosed herein. For example, procedures involving the right atrium may be performed in connection with treatment for inappropriate sinus tachycardia (e.g., crista line, inferior vena cava, and/or superior vena cava), atrial fibrillation (e.g., Cox maze lesions—right side), supraventricular tachycardia, and/or Wolff-Parkinson-White Syndrome. Procedures involving the right ventricle may be performed in connection with treatment for ventricular tachycardia (e.g., linear or spot lesions) (e.g., right ventricle posterior wall, right ventricle lateral free wall, right ventricle anterior, septum, right ventricle papillary muscles, and/or right ventricle outflow tract), partial ventricular contractors (e.g., right ventricle outflow tract septum, basal right ventricle, and/or right ventricle outflow tract free wall), and/or Brugada Syndrome (e.g., right ventricle outflow tract), for example. Procedures involving the left atrium may be performed in connection with treatment for atrial fibrillation (e.g., encircling or linear lesions) (e.g., ligament of Marshall, roof and floor lines, left atrium posterior wall, isthmus line, and/or autonomics (ganglionated plexus)), supra ventricular tachycardia, and/or left atrial appendage isolation (e.g., left atrial appendage ostium). Procedures involving the left ventricle may be performed in connection with syncope (e.g., autonomics (ganglionated plexus)), atrial tachycardia (e.g., anywhere in the left ventricle), atrial flutter (e.g., mitral valve), Wolff-Parkinson-White Syndrome (e.g., atrioventricular groove), partial ventricular contractions (e.g., left ventricle outflow tract and/or aortic root), hypertension (e.g., anywhere in the left ventricle), Brugada and/or ventricular tachycardia (e.g., linear or spot lesions) (e.g., left ventricle posterior wall, left ventricle lateral free wall, left ventricle anterior, septum, left ventricle papillary muscles, and/or left ventricle summit), for example. Procedures involving the right ventricle/left ventricle septum may be performed in connection with ventricular tachycardia (e.g., combined right ventricle and left ventricle lesion), for example. Some procedures may be performed involving the right atrium/left atrium septum. It will be understood that the foregoing list is merely exemplary and is not to be considered limiting.

The present disclosure contemplates that ablation systems configured to perform pulsed field ablation (“PFA”) may be used in various medical and surgical procedures. Generally, PFA systems may be used to ablate targeted cells while limiting potential collateral damage to non-targeted tissues. PFA typically involves applying high-voltage electrical pulses to a target tissue. The pulses create high-intensity electrical fields, which disrupt the integrity of the cell membranes in the target tissue. As a result, over a short period of time (e.g., days to weeks), the cells die, creating a lesion in the target tissue. The present disclosure contemplates that PFA may be used for ablation of cardiac tissue for treatment of cardiac arrhythmias. Generally, any ablation device according to at least some aspects of the present disclosure may be utilized in connection with radiofrequency, pulsed field ablation, and/or any other electrical ablation modality.

Some example embodiments according to at least some aspects of the present disclosure may be configured without a heat sink and/or without active cooling (e.g., open or closed-circuit liquid cooling). Some example embodiments, such as those configured for bipolar operation, may be configured without a monopolar ground (e.g., return electrode).

As used herein, including in the claims, “or” as used in a list of items (for example, a list of items prefaced by a phrase such as “at least one of” or “one or more of”) indicates an inclusive list such that, for example, a list of [at least one of A, B, or C] means A or B or C or AB or AC or BC or ABC (i.e., A and B and C).

As those of some skill in this art will by now appreciate and depending on the particular application at hand, many modifications, substitutions and variations can be made in and to the materials, apparatus, configurations and methods of use of the devices of the present disclosure without departing from the spirit and scope thereof. In light of this, the scope of the present disclosure should not be limited to that of the particular embodiments illustrated and described herein, as they are merely by way of some examples thereof, but rather, should be fully commensurate with that of the claims appended hereafter and their functional equivalents.

1. An electrosurgical system, comprising:
an electrosurgical device, including:
a first electrode;
a second electrode; and
an intermediate electrical element;
wherein the first electrode, the second electrode, and the intermediate electrical element are configured to electrically communicate with a target tissue; and
wherein the intermediate electrical element interposes the first electrode and the second electrode; and
a resistive voltage divider electrically connected to a first input conductor and a second input conductor, the resistive voltage divider comprising a first resistor and a second resistor;
wherein the first resistor and the second resistor are electrically connected in series between the first input conductor and the second input conductor; and
wherein the first electrode is configured to electrically connect to the first input connector, the second electrode is configured to electrically connect to the second input connector, and the at least one intermediate electrode is configured to electrically connect to at least one intermediate conductor electrically connected between the first resistor and the second resistor.

2. The electrosurgical system of claim 1,
wherein the resistive voltage divider is disposed within at least one of a handle, a shaft, an end effector, or a connecting element of the electrosurgical device;
wherein the first input conductor and the second input conductor are configured to releasably electrically couple to an electrosurgical generator;
wherein the first input conductor is electrically coupled to the first electrode;
wherein the second input conductor is electrically coupled to the second electrode; and
wherein the at least one intermediate electrode is electrically coupled to the at least one intermediate conductor.

3. The electrosurgical system of claim 1,
wherein the resistive voltage divider is disposed within an interface component configured to electrically interpose the electrosurgical device and the electrosurgical generator;
wherein the first input conductor and the second input conductor are configured to releasably electrically couple to an electrosurgical generator;
wherein the first input conductor is configured to releasably electrically couple to the first electrode;
wherein the second input conductor is configured to releasably electrically couple to the second electrode; and
wherein the at least one intermediate conductor is configured to releasably electrically couple to the at least one intermediate electrode.

4. The electrosurgical system of claim 2,
wherein the resistive voltage divider is disposed within the electrosurgical generator;
wherein the first input conductor is configured to releasably electrically couple to the first electrode;
wherein the second input conductor is configured to releasably electrically couple to the second electrode; and
wherein the at least one intermediate conductor is configured to releasably electrically couple to the at least one intermediate electrode.

5. An electrosurgical device, comprising:
a first electrode;
a second electrode; and
at least one intermediate electrically resistive element;
wherein the first electrode, the second electrode, and the at least one intermediate electrically resistive element are configured to electrically communicate with a target tissue; and
wherein the at least one intermediate electrically resistive element interposes the first electrode and the second electrode.

6. The electrosurgical device of claim 5,
wherein the at least one intermediate electrically resistive element comprises a first electrically resistive element electrically connected to the first electrode and a second electrically resistive element electrically connected to the second electrode; and
wherein the first electrically resistive element is not directly electrically connected to the second electrically resistive element.

7. The electrosurgical device of claim 6, wherein the first electrically resistive element and the second electrically resistive element are interposed by a gap.

8. The electrosurgical device of claim 7, wherein the gap comprises at least one of an unoccupied space and a non-conductive element.

9. The electrosurgical device of claim 5, wherein the at least one intermediate electrically resistive element is electrically connected between the first electrode and the second electrode.

10. The electrosurgical device of claim 5, wherein an electrical resistance of the at least one intermediate electrically resistive element is approximately equal to an electrical resistance of a target tissue.

11. An ablation device for creating a lesion in a target tissue, the ablation device comprising:
an end effector comprising
a tissue engagement portion configured to engage a target tissue, the tissue engagement portion configured for electrical contact with the target tissue and comprising a first tissue contact, a second tissue contact, and an intermediate tissue contact;
wherein the intermediate tissue contact is disposed between the first tissue contact and the second tissue contact; and
wherein the first tissue contact, the intermediate tissue contact, and the second tissue contact are electrically coupled so that, when the end effector is supplied with electrical ablation energy, a magnitude of at least one electrical parameter differs between the first tissue contact, the intermediate tissue contact, and the second tissue contact so that an intermediate tissue contact magnitude is between a first tissue contact magnitude and a second tissue contact magnitude.

12. The ablation device of claim 11, wherein the tissue engagement portion comprises a discrete first electrode comprising the first tissue contact and a discrete second electrode comprising the second tissue contact.

13. The ablation device of claim 12, wherein the tissue engagement portion comprises a discrete intermediate electrode comprising the intermediate tissue contact.

14. The ablation device of claim 13, wherein the tissue engagement portion comprises a first insulator between the first electrode and the intermediate electrode and a second insulator between the intermediate electrode and the second electrode.

15. The ablation device of claim 13,
wherein the intermediate electrode comprises at least two sequentially disposed, discrete intermediate electrodes; and
wherein the magnitude of the at least one electrical parameter differs incrementally between the at least two sequentially disposed, discrete intermediate electrodes.

16. The ablation device of claim 13,
wherein the first electrode, the intermediate electrode, and the second electrode are disposed in a line; and
wherein the first electrode is disposed as a first outermost electrode at a first end and the second electrode is disposed as a second outermost electrode at a second end.

17. The ablation device of claim 13, wherein the first electrode is nested within the intermediate electrode, and the intermediate electrode is nested within the second electrode.

18. A method of creating a lesion in a target tissue, the method comprising:
positioning a tissue engagement portion of an end effector of an ablation device proximate a target tissue so that a first tissue contact of the tissue engagement portion is in electrical contact with the target tissue, a second tissue contact of the tissue engagement portion is in electrical contact with the target tissue, and an intermediate tissue contact of the tissue engagement portion between the first tissue contact and the second tissue contact is in electrical contact with the target tissue;
creating a lesion in the target tissue by applying electrical ablation energy to the end effector so that a magnitude of at least one electrical parameter differs between the first tissue contact, the intermediate tissue contact, and the second tissue contact so that the magnitude at the intermediate tissue contact is less than the magnitude at the first tissue contact and greater than the magnitude at the second tissue contact.
